SEQUENCE LISTING (45 sequences)

SEQ ID NO: 1 - DNA, 24 bp, Homo sapiens
tcgtcgttttgtcgttttgtcgtt

SEQ ID NO: 2 - DNA, 20 bp, Homo sapiens
tccatgacgttcctgacgtt

SEQ ID NO: 3 - protein, 28 aa, Artificial sequence
  synthetic sequence
GCPRILMRCKQDSDCLAGCVCGPNGFCG

SEQ ID NO: 4 - protein, 34 aa, Artificial sequence
  synthetic sequence
CVRLHESCLGQQVPCCDPAATCYCRFFNAFCYCR

SEQ ID NO: 5 - protein, 29 aa, Artificial sequence
  synthetic sequence
CGETCVGGTCNTPGCTCSWPVCTRNGLPV

SEQ ID NO: 6 - protein, 34 aa, Artificial sequence
  synthetic sequence
SGSDGGVCPKILKKCRRDSDCPGACICRGNGYCG

SEQ ID NO: 7 - protein, 36 aa, Artificial sequence
  synthetic sequence
MCMPCFTTDHQMARKCDDCCGGKGRGKCYGPQCLCR

SEQ ID NO: 8 - protein, 33 aa, Artificial sequence
  synthetic sequence
GCPQGRGDWAPTSCKQDSDCRAGCVCGPNGFCG

SEQ ID NO: 9 - protein, 33 aa, Artificial sequence
  synthetic sequence
GCPRPRGDNPPLTCSQDSDCLAGCVCGPNGFCG

SEQ ID NO: 10 - protein, 33 aa, Artificial sequence
  synthetic sequence
GCPRPRGDNPPLTCZQDSDCLAGCVCGPNGYCG

SEQ ID NO: 11 - protein, 33 aa, Artificial sequence
  synthetic sequence
GCAEPRGDMPWTWCKQDSDCLAGCVCGPNGFCG

SEQ ID NO: 12 - protein, 33 aa, Artificial sequence
  synthetic sequence
GCVGGRGDWSPKWCKQDSDCPAGCVCGPNGFCG

SEQ ID NO: 13 - protein, 33 aa, Artificial sequence
  synthetic sequence
GCAELRGDRSYPECKQDSDCLAGCVCGPNGFCG

SEQ ID NO: 14 - protein, 33 aa, Artificial sequence
  synthetic sequence
GCRLPRGDVPRPHCKQDSDCQAGCVCGPNGFCG

SEQ ID NO: 15 - protein, 33 aa, Artificial sequence
  synthetic sequence
GCYPLRGDNPYAACKQDSDCRAGCVCGPNGFCG

SEQ ID NO: 16 - protein, 33 aa, Artificial sequence
  synthetic sequence
GCTIGRGDWAPSECKQDSDCLAGCVCGPNGFCG

SEQ ID NO: 17 - protein, 33 aa, Artificial sequence
  synthetic sequence
GCHPPRGDNPPVTCKQDSDCLAGCVCGPNGFCG

SEQ ID NO: 18 - protein, 33 aa, Artificial sequence
  synthetic sequence
GCPEPRGDNPPPSCKQDSDCRAGCVCGPNGFCG

SEQ ID NO: 19 - protein, 33 aa, Artificial sequence
  synthetic sequence
GCLPPRGDNPPPSCKQDSDCQAGCVCGPNGFCG

SEQ ID NO: 20 - protein, 33 aa, Artificial sequence
  synthetic sequence
GCHLGRGDWAPVGCKQDSDCPAGCVCGPNGFCG

SEQ ID NO: 21 - protein, 33 aa, Artificial sequence
  synthetic sequence
GCNVGRGDWAPSECKQDSDCPAGCVCGPNGFCG

SEQ ID NO: 22 - protein, 33 aa, Artificial sequence
  synthetic sequence
GCFPGRGDWAPSSCKQDSDCRAGCVCGPNGFCG

SEQ ID NO: 23 - protein, 33 aa, Artificial sequence
  synthetic sequence
GCPLPRGDNPPTECKQDSDCQAGCVCGPNGFCG

SEQ ID NO: 24 - protein, 33 aa, Artificial sequence
  synthetic sequence
GCSEARGDNPRLSCKQDSDCRAGCVCGPNGFCG

SEQ ID NO: 25 - protein, 33 aa, Artificial sequence
  synthetic sequence
GCLLGRGDWAPEACKQDSDCRAGCVCGPNGFCG

SEQ ID NO: 26 - protein, 33 aa, Artificial sequence
  synthetic sequence
GCHVGRGDWAPLKCKQDSDCQAGCVCGPNGFCG

SEQ ID NO: 27 - protein, 33 aa, Artificial sequence
  synthetic sequence
GCVRGRGDWAPPSCKQDSDCPAGCVCGPNGFCG

SEQ ID NO: 28 - protein, 33 aa, Artificial sequence
  synthetic sequence
GCLGGRGDWAPPACKQDSDCRAGCVCGPNGFCG

SEQ ID NO: 29 - protein, 33 aa, Artificial sequence
  synthetic sequence
GCFVGRGDWAPLTCKQDSDCQAGCVCGPNGFCG

SEQ ID NO: 30 - protein, 33 aa, Artificial sequence
  synthetic sequence
GCPVGRGDWSPASCKQDSDCRAGCVCGPNGFCG

SEQ ID NO: 31 - protein, 33 aa, Artificial sequence
  synthetic sequence
GCPRPRGDNPPLTCKQDSDCLAGCVCGPNGFCG

SEQ ID NO: 32 - protein, 33 aa, Artificial sequence
  synthetic sequence
GCYQGRGDWSPSSCKQDSDCPAGCVCGPNGFCG

SEQ ID NO: 33 - protein, 33 aa, Artificial sequence
  synthetic sequence
GCAPGRGDWAPSECKQDSDCQAGCVCGPNGFCG

SEQ ID NO: 34 - protein, 33 aa, Artificial sequence
  synthetic sequence
GCVQGRGDWSPPSCKQDSDCPAGCVCGPNGFCG

SEQ ID NO: 35 - protein, 33 aa, Artificial sequence
  synthetic sequence
GCHVGRGDWAPEECKQDSDCQAGCVCGPNGFCG

SEQ ID NO: 36 - protein, 33 aa, Artificial sequence
  synthetic sequence
GCDGGRGDWAPPACKQDSDCRAGCVCGPNGFCG

SEQ ID NO: 37 - protein, 33 aa, Artificial sequence
  synthetic sequence
GCPQGRGDWAPEWCKQDSDCPAGCVCGPNGFCG

SEQ ID NO: 38 - protein, 33 aa, Artificial sequence
  synthetic sequence
GCPRGRGDWSPPACKQDSDCQAGCVCGPNGFCG

SEQ ID NO: 39 - protein, 38 aa, Artificial sequence
  synthetic sequence
GCVRLHESCLGQQVPCCDPAATCYCSGRGDNDLVCYCR

SEQ ID NO: 40 - protein, 38 aa, Artificial sequence
  synthetic sequence
GCVRLHESCLGQQVPCCDPAATCYCKGRGDARLQCYCR

SEQ ID NO: 41 - protein, 38 aa, Artificial sequence
  synthetic sequence
GCVRLHESCLGQQVPCCDPAATCYCVGRGDDNLKCYCR

SEQ ID NO: 42 - protein, 38 aa, Artificial sequence
  synthetic sequence
GCVRLHESCLGQQVPCCDPAATCYCEGRGDRDMKCYCR

SEQ ID NO: 43 - protein, 38 aa, Artificial sequence
  synthetic sequence
GCVRLHESCLGQQVPCCDPAATCYCYGRGDNDLRCYCR

SEQ ID NO: 44 - protein, 15 aa, Artificial sequence
  synthetic sequence
GGGGSGGGGSGGGGS

SEQ ID NO: 45 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2022/0257785 A1
(43) Pub. Date: Aug. 18, 2022

(54) KNOTTIN-IMMUNOSTIMULANT CONJUGATES AND RELATED COMPOSITIONS AND METHODS

(71) Applicant: The Board of Trustees of the Leland Stanford Junior University, Stanford, CA (US)

(72) Inventors: Idit Sagiv-Barfi, Stanford, CA (US); Ronald Levy, Stanford, CA (US); Caitlyn Miller, Stanford, CA (US); Jennifer R. Cochran, Stanford, CA (US); Carolyn R. Bertozzi, Stanford, CA (US)

(21) Appl. No.: 17/762,694

(22) PCT Filed: Sep. 29, 2020
(86) PCT No.: PCT/US2020/053258

§ 371 Date: Mar. 22, 2022

Related U.S. Application Data
(60) Provisional application No. 62/908,305, filed on Sep. 30, 2019.

(51) Int. Cl. A61K 47/64; A61K 47/54; A61K 47/55; A61P 35/00

(52) U.S. Cl. CPC A61K 47/646; A61P 35/00; A61K 47/55; A61K 47/549

(57) ABSTRACT

Provided are conjugates comprising a knottin peptide comprising an engineered loop that binds to a cell surface molecule, and an immunostimulant conjugated to the knottin peptide via a linker. According to some embodiments, the immunostimulant activates a pathogen recognition receptor (PRR). For example, the immunostimulant may be a Toll-Like Receptor (TLR) agonist, e.g., an agonist of TLR 7, TLR 8 and/or TLR 9. Also provided are compositions (e.g., pharmaceutical compositions) that comprise the conjugates of the present disclosure, as well as kits comprising such compositions and methods of using such compositions, e.g., to treat an individual having cancer. Methods of making the conjugates of the present disclosure are also provided.

### CROSS-REFERENCE TO RELATED APPLICATIONS

This application claims the benefit of U.S. Provisional Patent Application No. 62/908,305, filed Sep. 30, 2019, which application is incorporated herein by reference in its entirety.

### INTRODUCTION

In situ cancer vaccination represents a therapeutic strategy involving intratumoral injection of immunoenhancing agents, such as Toll-Like Receptor (TLR) agonists, to trigger immune activation and exploit tumor-associated antigens available at the tumor site. The advantage of this approach is that virtually any type of cancer can be treated without prior knowledge of the specific tumor antigens. The main limitation of this strategy is the requirement for intratumoral injection, which is very challenging for cancers with limited accessibility (e.g. lung cancer, pancreatic cancer, renal cancer, etc.) and in cases of metastatic disease when the primary tumor has been surgically resected.

### SUMMARY

Provided are conjugates comprising a knottin peptide comprising an engineered loop that binds to a cell surface molecule, and an immunostimulant conjugated to the knottin peptide via a linker. According to some embodiments, the immunostimulant activates a pathogen recognition receptor (PRR). For example, the immunostimulant may be a Toll-Like Receptor (TLR) agonist, e.g., an agonist of TLR 7, TLR 8 and/or TLR 9. Also provided are compositions (e.g., pharmaceutical compositions) that comprise the conjugates of the present disclosure, as well as kits comprising such compositions and methods of using such compositions, e.g., to treat an individual having cancer. Methods of making the conjugates of the present disclosure are also provided.

### BRIEF DESCRIPTION OF THE FIGURES

FIG. 1: Schematic illustration of tumor-targeting immunostimulant conjugates using an integrin-binding knottin peptide and knottin-Fc fusion as targeting agents.

FIG. 2: Strategies to functionalize immunostimulants according to some embodiments of the present disclosure.

FIG. 3: Sequences and schematic illustrations of knottin peptides according to some embodiments of the present disclosure.

FIG. 4: Strategies to conjugate knottin peptides to functionalized immunostimulants according to some embodiments of the present disclosure.

FIG. 5: Knottin-CpG conjugates with CpG incorporated at different sites according to some embodiments of the present disclosure.

FIG. 6: Knottin-CpG conjugates synthesized with different linkers according to some embodiments of the present disclosure.

FIG. 7: Knottin-TLR7/8 agonist conjugates according to some embodiments of the present disclosure.

FIG. 8: A Knottin-Fc immunostimulant conjugate according to some embodiments of the present disclosure.

FIG. 9: A conjugate comprising a knottin peptide conjugated to a detectable label to enable in vivo fluorescence tracking according to some embodiments of the present disclosure.

FIG. 10: Data showing in vivo fluorescence imaging of a conjugate of the present disclosure administered intratumorally and peritumorally.

FIG. 11: Data showing in vivo fluorescence imaging of a conjugate of the present disclosure administered intravenously.

FIG. 12: Data showing ex vivo fluorescence imaging of a conjugate of the present disclosure in excised tumors.

FIG. 13: Survival curves demonstrating therapeutic efficacy of 3CM-CpG in 4T1-Luc breast carcinoma (n=9-10).

FIG. 14: Data showing 4T1-Luc average tumor growth over time.

FIG. 15: Data showing individual 4T1-Luc tumor growth curves.

FIG. 16: Provides immune cell infiltration data. Mice with 4T1-luc cell tumors were treated according to the schematic with intravenous (IV) or intratumoral (IT) injections for the following groups: Vehicle IV, CpG IV, 3CM-CpG IV, and CpG IT. Tumors were excised post-treatment and analyzed for tumor-infiltrating immune cells via FACS. Provided are plots showing the abundance of different immune populations as % of total alive single cells.

FIG. 17: Pie charts summarizing the average abundance (% total alive single cells) of immune cell populations and two uncharacterized cell populations (“Other”) for each treatment group described for FIG. 16.

### DETAILED DESCRIPTION

Before the conjugates, compositions, kits and methods of the present disclosure are described in greater detail, it is to be understood that the conjugates, compositions, kits and methods are not limited to particular embodiments described, as such may, of course, vary. It is also to be understood that the terminology used herein is for the purpose of describing particular embodiments only, and is not intended to be limiting, since the scope of the conjugates, compositions, kits and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the conjugates, compositions, kits and methods. The upper and lower limits of these smaller ranges may independently be included in the smaller ranges and are also encompassed within the conjugates, compositions, kits and methods, subject to any specifically excluded limit in the stated range. Where the stated range includes one or both of the limits, ranges excluding either or both of those included limits are also included in the conjugates, compositions, kits and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the conjugates, compositions, kits and methods belong. Although any conjugates, compositions, kits and methods similar or equivalent to those described herein can also be used in the practice or testing of the conjugates, compositions, kits and methods, representative illustrative conjugates, compositions, kits and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present conjugates, compositions, kits and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the conjugates, compositions, kits and methods, which are, for clarity, described in the context of separate embodiments, may also be provided in combination in a single embodiment. Conversely, various features of the conjugates, compositions, kits and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present conjugates, compositions, kits and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Conjugates

The present disclosure provides knottin peptide-immunostimulant conjugates. The conjugates comprise a knottin peptide comprising an engineered loop that binds to a cell surface molecule, and an immunostimulant conjugated to the knottin peptide via a linker. Such conjugates are useful in a variety of applications. For example, in the context of cancer therapy, and as demonstrated herein, the conjugates are unexpectedly capable of localizing to solid tumors following systemic administration and achieve substantially greater therapeutic efficacy than the corresponding non-conjugated immunostimulant. Moreover, the conjugates unexpectedly and significantly transform the tumor immune landscape compared to systemic administration of the immunostimulant not conjugated to the knottin peptide as indicated by an increased percentage of CD8+ T cells, CD4+ T cells, and B cells, as well as a decrease in myeloid-derived suppressor cells (MDSCs). This dramatic shift in immune cell profile using a knottin peptide-immunostimulant conjugate was indistinguishable from the shift observed from treatment with the immunostimulant administered intratumorally (IT). Details regarding the conjugates of the present disclosure will now be described.

By “immunostimulant” is meant a substance that directly or indirectly induces the activation or maturation of one or more types of cells of the immune system. Various types of immunostimulants may be provided in the conjugates of the present disclosure. Non-limiting examples of an immunostimulant that may be employed include a polypeptide, a nucleic acid (e.g., an oligonucleotide), a carbohydrate, an antibody, a ligand, an aptamer, a nanoparticle, and a small molecule. In some embodiments, the immunostimulant stimulates non-immune cells (e.g., epithelial cells, endothelial cells, tumor cells, etc.) to produce proinflammatory cytokines.

According to some embodiments, a conjugate of the present disclosure comprises an immunostimulant that directly or indirectly induces the activation or maturation of one or more types of cells of the innate immune system. Non-limiting examples of innate immune system cell types that may be directly or indirectly activated by the immunostimulant include macrophages, dendritic cells, NK cells, neutrophils, basophils, eosinophils, Langerhans cells, mast cells, and/or monocytes. In certain embodiments, a conjugate of the present disclosure comprises an immunostimulant that induces the activation or maturation of one or more types of cells of the adaptive immune system. Non-limiting examples of adaptive immune system cell types that may be activated by the immunostimulant include T cells and B cells. Examples of T cells include naive T cells (T_{N}), cytotoxic T cells (T_{CTL}), memory T cells (T_{MEM}), T memory stem cells (T_{SCM}), central memory T cells (T_{CM}), effector memory T cells (T_{EM}), tissue resident memory T cells (T_{RM}), effector T cells (T_{EFF}), regulatory T cells (T_{REGs}), helper T cells (T_{H}, T_{H}1, T_{H}2, T_{H}17) CD4+ T cells, CD8+ T cells, virus-specific T cells, alpha beta T cells (Tap), and gamma delta T cells (Ty).

In certain embodiments, the immunostimulant comprises a pathogen-associated molecular pattern (PAMP). PAMPs include pathogen-specific sugars, lipoproteins and/or nucleic acids (e.g., DNA comprising one or more unmethylated repeats of the dinucleotide CpG, double-stranded RNA (dsRNA), single-stranded RNA (ssRNA), or the like) expressed as part of the life cycle of a pathogen. Host proteins capable of recognizing such specific microbial patterns are called pathogen recognition receptors (PRRs). According to some embodiments, the immunostimulant of a conjugate of the present disclosure activates a pathogen recognition receptor (PRR). In certain embodiments, the PRR is selected from a Toll-like receptor (TLR), a RIG-1-like receptor (RLR), a nucleotide-binding oligomerization domain (NOD)-like receptor (NLR), a C-type lectin receptor (CLR), a cytosolic dsDNA sensor (CDS), a stimulator of interferon genes (STING), and any combination thereof. According to some embodiments, the immunostimulant activates a PRR and comprises a natural or non-natural PAMP. Non-natural PRR activators which may be employed include, but are not limited to, synthetic small molecule PRR agonists.

According to some embodiments, the immunostimulant is a Toll-Like Receptor (TLR) agonist. TLRs are a family of type I transmembrane PRRs that sense invading pathogens or endogenous damage signals and initiate the innate and adaptive immune response. There are ten functional TLRs in human (TLR1-10) and twelve in mice (TLR1-9, 11-13). Various combinations of TLRs are expressed by different subsets of immune and non-immune cell types such as monocytes, macrophages, dendritic cells, neutrophils, B cells, T cells, fibroblasts, endothelial cells, and epithelial cells. Of the human TLRs, TLR1, 2, 4, 5, 6, and 10 are expressed on the cell surface and primarily recognize microbial membrane and/or cell wall components, while TLR3, 7, 8, and 9 are expressed in the membranes of endolysosomal compartments and recognize nucleic acids. TLRs have a variable number of ligand sensing, leucine-rich repeats (LRR) at their N-terminal ends and a cytoplasmic Toll/IL-1 R (TIR) domain. The TIR domain mediates interactions between TLRs and adaptor proteins involved in regulating TLR signaling including MyD88, TRIF, TRAM, and TIRAP/MAL. Signaling pathways activated downstream of these adaptor molecules promote the expression of pro-inflammatory cytokines, chemokines, and type I and type Ill interferons.

In certain embodiments, a conjugate of the present disclosure comprises an immunostimulant which is an agonist of one or more of TLR1, TLR2, TLR3, TLR4, TLR5, TLR6, TLR7, TLR8, TLR9, and TLR10. According to some embodiments, the immunostimulant is a TLR 9 agonist. The human TLR9 gene can splice into different isoforms during transcription generating 5 TLR9 isoforms (TLR9A, B, C, D, and E). These TLR9 isoforms are differentially expressed in various immune organs and cells, such as spleen, peripheral blood mononuclear cells (PBMC), and lymph nodes.

In some embodiments, the immunostimulant is an oligonucleotide-based TLR 9 agonist. As used herein, an “oligonucleotide” is a single-stranded multimer of nucleotides from 5 to 500 nucleotides, e.g., 5 to 100 nucleotides. Oligonucleotides may be synthetic or may be made enzymatically, and, in some embodiments, are 5 to 50 nucleotides in length. Oligonucleotides may contain ribonucleotide monomers (i.e., may be oligoribonucleotides or “RNA oligonucleotides”), deoxyribonucleotide monomers (i.e., may be oligodeoxyribonucleotides or “DNA oligonucleotides”), or a combination thereof. Oligonucleotides may be 10 to 20, 20 to 30, 30 to 40, 40 to 50, 50 to 60, 60 to 70, 70 to 80, 80 to 100, 100 to 150 or 150 to 200, or up to 500 nucleotides in length, for example.

When the immunostimulant is an oligonucleotide-based TLR 9 agonist, the immunostimulant may be an oligonucleotide comprising one or more unmethylated CpG dinucleotides. The role that TLR9 plays in the innate immune responses to bacterial and synthetic DNA containing unmethylated CpG motifs has been well characterized. See, e.g., Uematsu S, Akira S. (2006) Journal of Molecular Medicine 84(9):712-725. According to some embodiments, when the immunostimulant is an oligonucleotide-based TLR 9 agonist, the immunostimulant is a CpG oligodeoxynucleotide (ODN). CpG ODNs are synthetic single-stranded DNA molecules containing unmethylated CpG dinucleotides in particular sequence contexts (CpG motifs) capable of activating leukocytes as evidenced in vitro and in vivo. Three major classes of CpG ODNs have been identified, based on their structural and biological characteristics, and are designated Class A, Class B, and Class C. Class A oligos, which feature a central palindromic CpG-containing phosphodiester (PO) structure followed by a phosphorothioate (PS) homopolymeric G-stretch, are robust inducers of interferon-α (IFN-α) production and dendritic cell maturation. Class B oligos, in contrast, usually contain a full phosphorothioate (PS) backbone. These oligos also stimulate IFN-α production, but to a lesser extent. However, they strongly activate B cells. Class C oligos combine the properties of Class A and B, and are characterized by their complete PS backbone and palindromic CpG-containing motifs. CpG ODNs contain one or more unmethylated CpG dinucleotides in specific sequence contexts, which are readily recognized by mammalian cells as an indication of microbial invasion, due to the rarity of this structure in mammalian genomes. In certain embodiments, a conjugate of the present disclosure includes a CpG ODN from a class selected from class A (type D), class B (type K), and class C.

In some embodiments, when the immunostimulant is an oligonucleotide-based TLR 9 agonist comprising one or more unmethylated CpG dinucleotides (e.g., a CpG ODN), the immunostimulant comprises at least 5 nucleotides. In some embodiments, the immunostimulant comprises from 2 to 100, e.g., about 8 to about 40, nucleotides. In some embodiments, the immunostimulant comprise 10 to 30 nucleotides. In some embodiments, the immunostimulant comprises 15-25 nucleotides. In some embodiments, the immunostimulant is a T-rich oligonucleotide that contains one or more poly T sequences and/or has greater than about 25% T nucleotide residues. In some embodiments, the immunostimulant has a GTC trinucleotide in place of the CG dinucleotide. In some embodiments, the immunostimulant has one or more modified cytosines. In some embodiments, the immunostimulant is a deoxyribonucleic acid molecule that is partially single-stranded, dumbbell-shaped, and covalently closed. In some embodiments, the immunostimulant comprises one or more of the following structures: [CGN]ₓ, [NaCG]ₓ, [NₐCGNb]ₓ, [NaCGTTNb]ₓ, and [NₐCGN_{b}CGN_{c}]ₓ, where N is any nucleotide base, x is 0-25 and a, b and c are independently 1-15. For example, sequences which fall within [NₐCGN_{b}]ₓ include ACGT, GTCGTT, TCGGTT, TGACGTT, and ACGTACGT.

CpG motifs exhibit species-specificity. For example, the optimal mouse CpG motif is GACGTT, while that for use in human contexts is GTCGTT. A Class B CpG ODN, CpG ODN 1826, is a well-defined murine TLR 9 agonist, and is thus widely used in rodent models. This oligo is effective in eliciting mouse B cell proliferation, maturation of antigen-presenting cells, and a polarized Th1-type cell response. CpG ODN 1826 contains 2 CpG dinucleotides, both flanked by -GA at the 5 end and -TT at the 3′ end. Its backbone is fully phosphothioated, providing nuclease resistance, as opposed to the natural PO backbone found in bacterial or viral genomes.

In certain embodiments, the oligonucleotide-based TLR 9 agonist is a human CpG ODN. Such a human CpG ODN may include the CpG motif GTCGTT. A non-limiting example of such a human CpG ODN has the sequence TCGTCGTTTTGTCGTTTTGTCGTT (SEQ ID NO:1). According to some embodiments, the oligonucleotide-based TLR 9 agonist is a mouse CpG ODN. Such a mouse CpG ODN may include the CpG motif GACGTT. A non-limiting example of such a mouse CpG ODN has the sequence TCCATGACGTTCCTGACGTT (SEQ ID NO:2).

An oligonucleotide-based immunostimulant may include one or more modification, e.g., to decrease or prevent nuclease sensitivity. Examples of such modifications include modifications into native phosphodiester oligodeoxyribonucleotide and ribonucleotide polymers. For example, an oligonucleotide-based immunostimulant may include one or more phosphorothioate (PS) bonds. The PS bond substitutes a sulfur atom for a non-bridging oxygen in the phosphate backbone of an oligonucleotide. PS modification renders the internucleotide linkage more resistant to nuclease degradation.

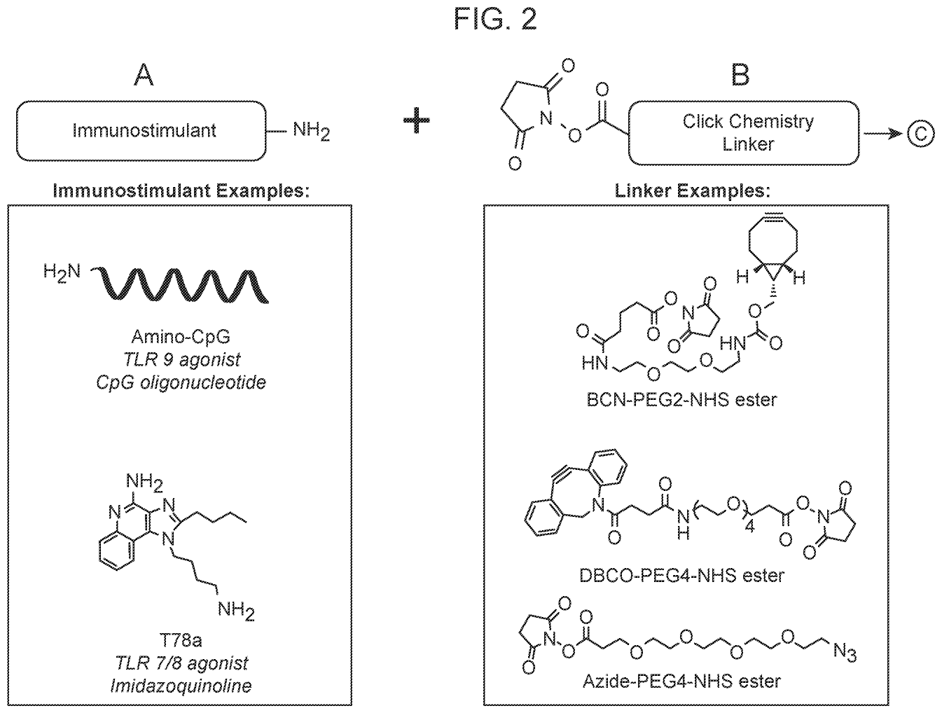

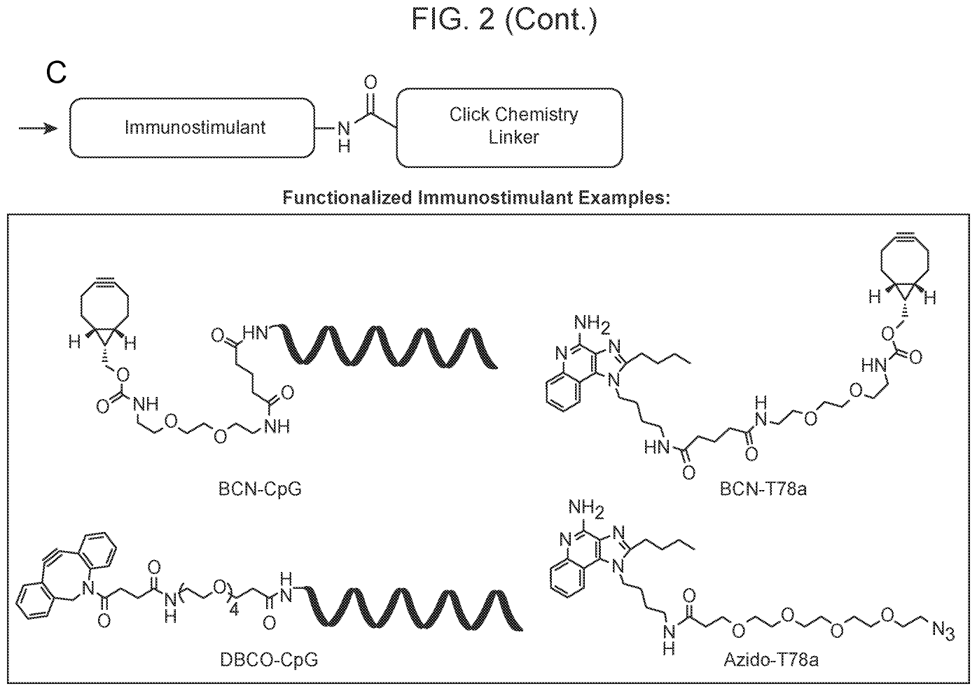

According to some embodiments, the immunostimulant is a TLR 7 agonist, a TLR 8 agonist, or both. A wide variety of TLR 7 and/or TLR 8 agonists are known. In certain embodiments, the TLR 7 and/or TLR 8 agonist comprises an imidazoquinoline (IMZQ) compound. Imidazoquinolines are powerful immunostimulants that function through Toll-like receptors, particularly TLR7 and TLR8. In certain embodiments, the TLR 7 and/or TLR 8 agonist that comprises an IMZQ compound is T78a, the structure of which is provided in FIG. 2. According to some embodiments, the TLR 7 and/or TLR 8 agonist that comprises an IMZQ compound is Hybrid-2 (1-(4-amino-2-butyl-1H-imidazo [4, 5-c] quinolin-1-yl)-2-methylpropan-2-ol); XG1-236 (2-butyl-2H-pyrazolo [3, 4-c] quinolin-4-amine); DS802 (2-butyl [1, 3] oxazolo [4, 5-c] quinolin-4-amine); CL075 (2-propyl [1, 3] thiazolo [4, 5-c]quinolin-4-amine); CL097 (2-(ethoxymethyl)-1H-imidazo [4, 5-c] quinolin-4-amine); R848 (1-[4-amino-2-(ethoxymethyl)-1H-imidazo [4, 5-c] quinolin-1-yl]-2-methylpropan-2-ol); Meta-amine, or Para-amine. See, e.g., Kubli-Garfias et al. (2017) PLoS ONE 12(6):e0178846; and Ganapathi et al. (2015) PLoS ONE 10(8):e0134640.

In certain embodiments, a conjugate of the present disclosure comprises two or more immunostimulants conjugated to the knottin peptide. For example, the knottin peptide may be conjugated to 2 or more, 3 or more, 4 or more, 5 or more, 6 or more, 7 or more, 8 or more, 9 or more, or 10 or more immunostimulants. When the knottin peptide is conjugated to two or more immunostimulants, two of the two or more immunostimulants may be the same or different. According to some embodiments, the two or more immunostimulants are independently selected from any of the immunostimulants (e.g., TLR 9 agonists, TLR 7 and/or 8 agonists, and/or the like) described herein.

According to some embodiments, the knottin peptide of a conjugate of the present disclosure is further conjugated to a detectable label. By “detectable label” is meant an agent that labels knottin peptide, such that the conjugate may be detected in an application of interest (e.g., in vitro and/or in vivo research and/or clinical applications). Detectable labels of interest include fluorescent labels (e.g., an AlexaFluor fluorophore, such as AlexaFluor 680 described in the Experimental section herein), radioisotopes, enzymes that generate a detectable product (e.g., horseradish peroxidase, alkaline phosphatase, luciferase, etc.), fluorescent proteins, paramagnetic atoms, and the like. In certain aspects, the knottin peptide is conjugated to a specific binding partner of detectable label, e.g., conjugated to biotin such that detection may occur via a detectable label that includes avidin/streptavidin.

In certain embodiments, the detectable label finds use in in vivo imaging, such as near-infrared (NIR) optical imaging, single-photon emission computed tomography (SPECT)/CT imaging, positron emission tomography (PET), nuclear magnetic resonance (NMR) spectroscopy, or the like. Detectable labels that find use in such applications include, but are not limited to, fluorescent labels, radioisotopes, and the like. In certain aspects, the detectable label is a multi-modal in vivo imaging agent that permits in vivo imaging using two or more imaging approaches (e.g., see Thorp-Greenwood and Coogan (2011) Dalton Trans. 40:6129-6143).

In certain embodiments, the detectable label is an in vivo imaging agent that finds use in near-infrared (NIR) imaging applications. Such agents include, but are not limited to, a Kodak X-SIGHT dye, Pz 247, DyLight 750 and 800 Fluors, Cy 5.5 and 7 Fluors, Alexa Fluor 680 and 750 Dyes, IRDye 680 and 8000W Fluors. According to some embodiments, the detectable label is an in vivo imaging agent that finds use in SPECT imaging applications, non-limiting examples of which include ^{99m}Tc, ¹¹¹In, ¹²³In, ²⁰¹Tl, and ¹³³Xe. In certain embodiments, the detectable label is an in vivo imaging agent that finds use in positron emission tomography (PET) imaging applications, e.g., ¹¹C, ¹³N, ¹⁵O, ¹⁸F, ⁶⁴Cu, ⁶²Cu, ¹²⁴I, ⁷⁶Br, ⁸²Rb, ⁶⁸Ga, or the like.

Knottin Peptides

The conjugates of the present disclosure include a knottin peptide that includes an engineered loop that binds to a cell surface molecule. The type of knottin peptide employed in the conjugates of the present disclosure may vary. Non-limiting examples of a knottin peptide that may be employed include an EETI-II peptide, an AgRP peptide, a w-conotoxin peptide, a Kalata B1 peptide, an MCoTI-II peptide, an agatoxin peptide, and a chlorotoxin peptide. The three-dimensional structure of a knottin peptide is minimally defined by a particular arrangement of three disulfide bonds. This characteristic topology forms a molecular knot in which one disulfide bond passes through a macrocycle formed by the other two intra-chain disulfide bridges. Although their secondary structure content is generally low, knottins share a small triple-stranded antiparallel β-sheet, which is stabilized by the disulfide bond framework. Folding and functional activity of knottins are often mediated by loop regions that are diverse in both length and amino acid composition. While three disulfide bonds are the minimum number that defines the fold of this family of peptides, knottins can also contain additional cysteine residues, yielding molecules with four or more disulfide bonds and additional constrained loops in their structure. The term “cystine” refers to a Cys residue in which the sulfur group is linked to another amino acid though a disulfide linkage; the term “cysteine” refers to the —SH (“half cystine”) form of the residue. Binding loop portions may be adjacent to cystines, such that there are no other intervening cystines in the primary sequence in the binding loop.

The knottin peptide may be a peptide described in the online KNOTTIN database, which includes detailed amino acid sequence, structure, classification and function information for thousands of polypeptides identified as contain cystine-knot motifs. Knottins are found in a variety of plants, animals, insects and fungi.

The knottin peptide may be full-length (that is, the length of the wild-type peptide/polypeptide), the knottin peptide may be truncated relative to the length of the wild-type peptide/polypeptide, or the knottin peptide may include additional amino acids such that the peptide is greater in length relative to the length of the wild-type peptide/polypeptide.

According to certain embodiments, a knottin-drug conjugate (KDC) of the present disclosure includes a knottin peptide based on any one of an Ecballium elaterium trypsin inhibitor II (EETI-II) peptide, an agouti-related protein (AgRP) peptide, a ω-conotoxin peptide, a Kalata B1 peptide, an MCoTI-II peptide, an agatoxin peptide, or a chlorotoxin peptide. In some embodiments, the knottin peptide is based on an Ecballium elaterium trypsin inhibitor II (EETI-II) peptide. In some embodiments, the knottin peptide is based on an agouti-related protein (AgRP) peptide.

By “EETI” is meant Protein Data Bank Entry (PDB) 2ETI. Its entry in the KNOTTIN database is EETI-II. In certain aspects, a knottin peptide of a conjugate of the present disclosure is based on an EETI-II peptide having the following amino acid sequence:

(SEQ ID NO: 3)
GCPRILMRCKQDSDCLAGCVCGPNGFCG

By “AGRP” is meant PDB entry 1 HYK and KNOTTIN database entry SwissProt AGRP_HUMAN. AGRP is a 132 amino acid neuropeptide that binds to melanocortin receptors in the human brain and is involved in regulating metabolism and appetite. The biological activity of AgRP is mediated by its C-terminal cysteine knot domain, which contains five disulfide bonds, but a fully active 34 amino acid truncated AgRP that contains only four disulfide bonds has been developed. In certain aspects, a knottin peptide of a conjugate of the present disclosure is based on a truncated AGRP peptide having the following amino acid sequence:

(SEQ ID NO: 4)
CVRLHESCLGQQVPCCDPAATCYCRFFNAFCYCR

According to some embodiments, a knottin peptide of a conjugate of the present disclosure is based on a Kalata B1 peptide having the following amino acid sequence:

(SEQ ID NO: 5)
CGETCVGGTCNTPGCTCSWPVCTRNGLPV

In certain embodiments, a knottin peptide of a conjugate of the present disclosure is based on a MCoTI-II peptide having the following amino acid sequence:

(SEQ ID NO: 6)
SGSDGGVCPKILKKCRRDSDCPGACICRGNGYCG

According to some embodiments, a knottin peptide of a conjugate of the present disclosure is based on a chlorotoxin peptide having the following amino acid sequence:

(SEQ ID NO: 7)
MCMPCFTTDHQMARKCDDCCGGKGRGKCYGPQCLCR

Sequences and structural (e.g., loop) information for EETI-II, AgRP, w-conotoxin, Kalata B1, MCoTI-II, agatoxin, chlorotoxin, and other knottin peptides upon which the knottin peptides of the conjugates of the present disclosure may be based may be found in the PDB, the KNOTTIN database, and other protein databases.

The knottin peptide includes an engineered loop that binds to a cell surface molecule—that is, the loop is engineered to bind to a target molecule on the surface of a cell. Knottins contain three disulfide bonds interwoven into a molecular ‘knot’ that constrain loop regions to a core of anti-parallel β-sheets. Wild-type EETI, for example, is composed of 28 amino acids with three disulfide-constrained loops: loop 1 (the trypsin binding loop, residues 3-8), loop 2 (residues 10-14), and loop 3 (residues 22-26) Knottin family members, which include protease inhibitors, toxins, and antimicrobials, share little sequence homology apart from their core cysteine residues. As a result, their disulfide-constrained loops tolerate much sequence diversity, making knottins amenable for protein engineering applications where mutations need to be introduced into a protein without abolishing its three-dimensional fold.

The engineered loop may include amino acid substitutions, insertions, and/or deletions in an existing loop of the knottin peptide, or the engineered loop may be a loop added to the knottin protein. That is, the knottin peptide of the conjugate may include a loop in addition to the one or more loops present in the wild-type peptide. By combining directed evolution with computational covariance analysis, guidelines for introducing modifications (both in amino acid sequence and loop length) into the loop regions of the knottin scaffold have been elucidated. See, e.g., Lahti et al. (2009) PLoS Comput. Biol. 5(9): e1000499.

In some embodiments, the loop of the knottin is engineered to bind to a cancer cell surface molecule. By “cancer cell” is meant a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a liquid tumor, a primary tumor, a metastatic tumor, and the like. Such an engineered loop confers upon the knottin peptide a cancer cell surface molecular recognition property that is not present in the wild-type peptide. In certain aspects, the cancer is a cancer known to have one or more tumor antigens. Non-limiting examples of tumor antigens to which the engineered loop of the knottin may bind include 5T4, AXL receptor tyrosine kinase (AXL), B-cell maturation antigen (BCMA), c-MET, C4.4a, carbonic anhydrase 6 (CA6), carbonic anhydrase 9 (CA9), Cadherin-6, CD19, CD22, CD25, CD27L, CD30, CD33, CD37, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), ephrin A4 (EFNA4), epidermal growth factor receptor (EGFR), EGFRvIII, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), glycoprotein non-metastatic B (GPNMB), guanylate cyclase 2C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), and trophoblast cell-surface antigen (TROP-2).

According to some embodiments, the cell surface molecule to which the engineered loop of the knottin binds is a receptor, e.g., a cell adhesion receptor, a receptor for a soluble factor (e.g., a growth factor, chemokine, or other soluble factor receptor), an immune cell receptor, or the like. According to some embodiments, the cell surface molecule to which the engineered loop of the knottin binds is a cell adhesion receptor, e.g., a cell adhesion receptor (e.g., an integrin) expressed on the surface of cancer cells, expressed on the surface of tumor vasculature cells, and/or the like. In certain embodiments, when the receptor is a cell adhesion receptor, the receptor is an integrin. For example, a conjugate of the present disclosure may include a knottin peptide having a loop engineered to bind to any one of αvμ1 integrin, αvμ3 integrin, αvμ5 integrin, αvμ6 integrin, α5β1 integrin, or any combination thereof. According to certain embodiments, the engineered loop binds to each of αvμ1 integrin, αvμ3 integrin, αvμ5 integrin, αvμ6 integrin, and α5β1 integrin.

An EETI-based knottin peptide (designated EETI-2.5D) having an engineered binding loop that binds to each of αvμ1 integrin, αvμ3 integrin, αvμ5 integrin, αvμ6 integrin, and α5β1 integrin, which may be employed in a conjugate of the present disclosure, has the following amino acid sequence (with the integrin-binding loop underlined):

(SEQ ID NO: 8)
GCPQGRGDWAPTSCKQDSDCRAGCVCGPNGFCG

An EETI-based knottin peptide (designated EETI-2.5F) having an engineered binding Loop that binds to each of αvβ1 integrin, αvβ3 integrin, αvβ5 integrin, αvβ6 integrin, and α5β1 integrin, which may be employed in a conjugate of the present disclosure, has the following amino acid sequence (with the integrin-binding loop underlined):

(SEQ ID NO: 9)
GCPRPRGDNPPLTCSQDSDCLAGCVCGPNGFCG

An EETI-based knottin peptide (designated 3CM) having an engineered binding loop that binds to each of αvβ1 integrin, αvβ3 integrin, αvβ5 integrin, αvβ6 integrin, and α5β1 integrin, which may be employed in a conjugate of the present disclosure, has the following amino acid sequence (with the integrin-binding loop underlined), where Z=5-azido-L-norvaline:

(SEQ ID NO: 10)
GCPRPRGDNPPLTCZQDSDCLAGCVCGPNGYCG

In some embodiments, the knottin peptide of a conjugate of the present disclosure is an integrin-binding EETI-based knottin peptide as set forth in Table 1.

TABLE 1
Example EETI Intedrin-Binding Knottin Peptides

| Peptide | | |
|---|---|---|
| identifier | Sequence | SEQ ID NO: |
| 1.4A | GCAEPRGDMPWTWCKQDSDCLAGCVCGPNGFCG | (SEQ ID NO: 11) |
| 1.4B | GCVGGRGDWSPKWCKQDSDCPAGCVCGPNGFCG | (SEQ ID NO: 12) |
| 1.4C | GCAELRGDRSYPECKQDSDCLAGCVCGPNGFCG | (SEQ ID NO: 13) |
| 1.4E | GCRLPRGDVPRPHCKQDSDCQAGCVCGPNGFCG | (SEQ ID NO: 14) |
| 1.4H | GCYPLRGDNPYAACKQDSDCRAGCVCGPNGFCG | (SEQ ID NO: 15) |
| 1.5B | GCTIGRGDWAPSECKQDSDCLAGCVCGPNGFCG | (SEQ ID NO: 16) |
| 1.5F | GCHPPRGDNPPVTCKQDSDCLAGCVCGPNGFCG | (SEQ ID NO: 17) |
| 2.3A | GCPEPRGDNPPPSCKQDSDCRAGCVCGPNGFCG | (SEQ ID NO: 18) |
| 2.3B | GCLPPRGDNPPPSCKQDSDCQAGCVCGPNGFCG | (SEQ ID NO: 19) |
| 2.3C | GCHLGRGDWAPVGCKQDSDCPAGCVCGPNGFCG | (SEQ ID NO: 20) |
| 2.3D | GCNVGRGDWAPSECKQDSDCPAGCVCGPNGFCG | (SEQ ID NO: 21) |
| 2.3E | GCFPGRGDWAPSSCKQDSDCRAGCVCGPNGFCG | (SEQ ID NO: 22) |
| 2.3F | GCPLPRGDNPPTECKQDSDCQAGCVCGPNGFCG | (SEQ ID NO: 23) |
| 2.3G | GCSEARGDNPRLSCKQDSDCRAGCVCGPNGFCG | (SEQ ID NO: 24) |
| 2.3H | GCLLGRGDWAPEACKQDSDCRAGCVCGPNGFCG | (SEQ ID NO: 25) |
| 2.3I | GCHVGRGDWAPLKCKQDSDCQAGCVCGPNGFCG | (SEQ ID NO: 26) |
| 2.3J | GCVRGRGDWAPPSCKQDSDCPAGCVCGPNGFCG | (SEQ ID NO: 27) |
| 2.4A | GCLGGRGDWAPPACKQDSDCRAGCVCGPNGFCG | (SEQ ID NO: 28) |
| 2.4C | GCFVGRGDWAPLTCKQDSDCQAGCVCGPNGFCG | (SEQ ID NO: 29) |
| 2.4D | GCPVGRGDWSPASCKQDSDCRAGCVCGPNGFCG | (SEQ ID NO: 30) |
| 2.4E | GCPRPRGDNPPLTCKQDSDCLAGCVCGPNGFCG | (SEQ ID NO: 31) |
| 2.4F | GCYQGRGDWSPSSCKQDSDCPAGCVCGPNGFCG | (SEQ ID NO: 32) |
| 2.4G | GCAPGRGDWAPSECKQDSDCQAGCVCGPNGFCG | (SEQ ID NO: 33) |
| 2.4J | GCVQGRGDWSPPSCKQDSDCPAGCVCGPNGFCG | (SEQ ID NO: 34) |
| 2.5A | GCHVGRGDWAPEECKQDSDCQAGCVCGPNGFCG | (SEQ ID NO: 35) |
| 2.5C | GCDGGRGDWAPPACKQDSDCRAGCVCGPNGFCG | (SEQ ID NO: 36) |
| 2.5D | GCPQGRGDWAPTSCKQDSDCRAGCVCGPNGFCG | (SEQ ID NO: 8) |
| 2.5F | GCPRPRGDNPPLTCKQDSDCLAGCVCGPNGFCG | (SEQ ID NO: 9) |
| 3CM | GCPRPRGDNPPLTCZQDSDCLAGCVCGPNGYCG | (SEQ ID NO: 10) |
| 2.5H | GCPQGRGDWAPEWCKQDSDCPAGCVCGPNGFCG | (SEQ ID NO: 37) |
| 2.5J | GCPRGRGDWSPPACKQDSDCQAGCVCGPNGFCG | (SEQ ID NO: 38) |

In some embodiments, the knottin peptide of a conjugate of the present disclosure is an integrin-binding AgRP-based knottin peptide as set forth in Table 2.

TABLE 2
Example AqRP Intearin-Binding Knottin Peptides

| Clone | Loop 4 sequence |
|---|---|
| 7A (5E) (SEQ | GCVRLHESCLGQQVPCCDPAATCYCSGRGDNDLV |
| ID NO: 39) | CYCR |
| 7B (SEQ ID NO: | GCVRLHESCLGQQVPCCDPAATCYCKGRGDARLQ |
| 40) | CYCR |
| 7E (SEQ ID NO: | GCVRLHESCLGQQVPCCDPAATCYCVGRGDDNLK |
| 41) | CYCR |
| 7J (6B) (SEQ | GCVRLHESCLGQQVPCCDPAATCYCEGRGDRDMK |
| ID NO: 42) | CYCR |
| 7C (SEQ ID | GCVRLHESCLGQQVPCCDPAATCYC YGRGDNDLR |
| NO: 43) | CYCR |

According to some embodiments, the knottin peptide of a conjugate of the present disclosure comprises an engineered loop that binds to a protease. Non-limiting examples of proteases include membrane proteases, e.g., matriptase.

In some embodiments, the knottin peptide includes one or more unnatural amino acids. Such one or more unnatural amino acids may find use, e.g., to facilitate conjugation of the drug to the knottin peptide. Unnatural amino acids which find use, e.g., for preparing the conjugates of the present disclosure, include those having a functional group selected from an azide, alkyne, alkene, amino-oxy, hydrazine, aldehyde, nitrone, nitrile oxide, cyclopropene, norbornene, iso-cyanide, aryl halide, and boronic acid functional group. Unnatural amino acids which may be incorporated into a knottin peptide of a knottin-drug conjugate of the present disclosure, which unnatural amino acid may be selected to provide a functional group of interest are known and described in, e.g., Maza et al. (2015) Bioconjug. Chem. 26(9):1884-9; Patterson et al. (2014) ACS Chem. Biol. 9:592-605; Adumeau et al. (2016) Mol. Imaging Biol. (2):153-65; and elsewhere. In certain embodiments, the knottin peptide includes one or more 5-azido-L-norvaline residues or a derivative thereof, e.g., a derivative produced upon conjugation of the residue to a functionalized immunostimulant.

A conjugate of the present disclosure may comprise a knottin peptide having 70% or greater, 80% or greater, 90% or greater, 95% or greater, or 100% identity to the amino acid sequence of a knottin peptide described herein, e.g., any of the knottin peptide sequences provided in Table 1 or Table 2 above.

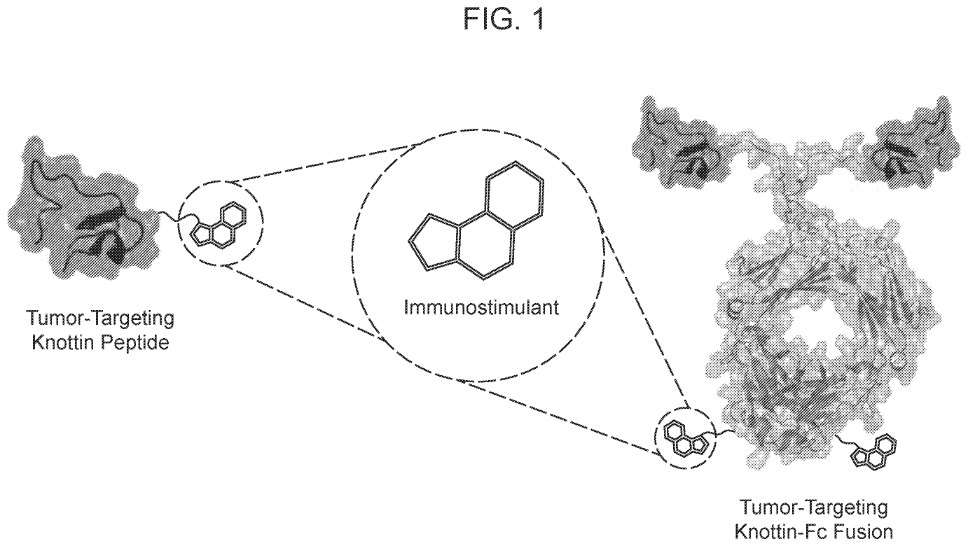

According to some embodiments, the knottin peptide of a conjugate of the present disclosure is fused to one or more heterologous polypeptides. The knottin peptide may be fused directly to a heterologous polypeptide. In certain embodiments, the knottin peptide is fused directly to a heterologous polypeptide via a linker. A non-limiting example of a linker that may be employed is a a serine-glycine linker, such as a serine-glycine linker that includes the amino acid sequence GGGGSGGGGSGGGGS (G₄S)₃ (SEQ ID NO:44). Heterologous polypeptides of interest include, but are not limited to, an Fc domain (e.g., a human or mouse Fc domain), an albumin, a transferrin, XTEN, a homo-amino acid polymer, a proline-alanine-serine polymer, an elastin-like peptide, or any combination thereof. In some embodiments, the heterologous polypeptide increases the stability and/or serum half-life of the knottin peptide upon its administration to an individual in need thereof, as compared to the same knottin peptide which is not fused to the heterologous polypeptide. In certain embodiments, provided are fusion proteins that include any of the knottin peptides of the present disclosure fused to a human Fc domain (e.g., a full-length human Fc domain or fragment thereof). A schematic illustration of a conjugate comprising a knottin peptide fused to an Fc domain and conjugated to an immunostimulant according to some embodiments of the present disclosure is schematically illustrated in FIG. 1. According to some embodiments, such a fusion protein finds use, e.g., in administering to an individual in need thereof in accordance with the methods of the present disclosure, e.g., an individual having cancer. A non-limiting example of a human Fc domain that may be fused to a knottin peptide of a conjugate of the present disclosure is a human IgG1 Fc domain having the sequence set forth in Table 3 below (SEQ ID NO:45), or a fragment thereof.

TABLE 3
Amino Acid Sequence of an Example Human Fc Domain

| | Amino Acid Sequence |
|---|---|
| Example Human | DKTHTCPPCPAPELLGGPSVFLFPPKPKDTLMISR |
| Fc Domain | TPEVTCVVVDVSHEDPEVKFNWYVDGVEVHNAKTK |
| (SEQ ID NO: 45) | PREEQYNSTYRVVSVLTVLHQDWLNGKEYKCKVSN |
| | KALPAPIEKTISKAKGQPREPQVYTLPPSRDELTK |
| | NQVSLTCLVKGFYPSDIAVEWESNGQPENNYKTTP |
| | PVLDSDGSFFLYSKLTVDKSRWQQGNVFSCSVMHE |
| | ALHNHYTQKSLSLSPGK |

In some embodiments, when the knottin peptide is fused to one or more heterologous polypeptides, the knottin peptide is fused to a heterologous polypeptide detectable in vivo. Non-limiting examples of in vivo detectable polypeptides include bioluminescence reporters. In certain embodiments, the bioluminescence reporter is a luciferase, e.g., a nanoluciferase.

The manner in which the knottin peptide having an engineered loop that binds to a cell surface molecule is developed may vary. Rational and combinatorial approaches have been used to engineer knottins with novel molecular recognition properties. For example, a library of knottin proteins may be created and screened, e.g., by bacterial display, phage display, yeast surface display, fluorescence-activated cell sorting (FACS), and/or any other suitable screening method.

Yeast surface display is a powerful combinatorial technology that has been used to engineer proteins with novel molecular recognition properties, increased target binding affinity, proper folding, and improved stability. In this platform, libraries of protein variants are generated and screened in a high-throughput manner to isolate mutants with desired biochemical and biophysical properties. Yeast surface display has proven to be a successful combinatorial method for engineering knottins with altered molecular recognition. Yeast surface display benefits from quality control mechanisms of the eukaryotic secretory pathway, chaperone-assisted folding, and efficient disulfide bond formation.

One example approach for developing a knottin peptide having an engineered loop that binds to a cell surface molecule of interest involves genetically fusing the peptide to the yeast mating agglutinin protein Aga2p, which is attached by two disulfide binds to the yeast cell wall protein Aga1p. This Aga2p-fusion construct, and a chromosomally integrated Aga1p expression cassette, may be expressed under the control of a suitable promoter, such as a galactose-inducible promoter. N- or C-terminal epitope tags may be included to measure cell surface expression levels by flow cytometry using fluorescently labeled primary or secondary antibodies. This construct represents the most widely used display format, where the N-terminus of the knottin (or other protein to be engineered) is fused to Aga2, but several alternative variations of the yeast surface display plasmid have been described and may be employed to develop a knottin peptide for use in a conjugate of the present disclosure. One of the benefits of this screening platform over panning-based methods used with phage or mRNA display is that two-color FACS can be used to quantitatively discriminate clones that differ by as little as two-fold in binding affinity to the desired target.

To selectively mutate knottin loop regions at the DNA level, degenerate codons can be introduced by oligonucleotide assembly using, e.g., overlap extension PCR. Next, the genetic material may be amplified using flanking primers with sufficient overlap with the yeast display vector for homologous recombination in yeast. This assembly and amplification method allow knottin libraries to be created at relatively low cost and effort. Synthetic oligonucleotide libraries and recent methods have been developed that allow defined control over library composition.

In certain aspects, a display library (e.g., a yeast display library) is screened for binding to the cell surface molecule of interest by FACS. When screening knottin libraries by FACS, an enriched pool of binders generally emerges in 4-7 rounds of sorting. Two-color FACS may be used for library screening, where one fluorescent label can be used to detect the c-myc epitope tag and the other to measure interaction of the knottin mutant against the binding target of interest. Different instrument lasers and/or filter sets can be used to measure excitation and emission properties of the two fluorophores at single-cell resolution. This enables yeast expression levels to be normalized with binding. That is, a knottin that exhibits poor yeast expression but binds a high amount of a target can be distinguished from a knottin that is expressed at high levels but binds weakly to a target. Accordingly, a two-dimensional flow cytometry plot of expression versus binding will result in a diagonal population of yeast cells that bind to target antigen. High-affinity binders can be isolated using library sort gates. Alternatively, in an initial sort round it could be useful to clear the library of undesired clones that do not express full-length. The target used in the screening is structurally and functionally relevant for the final application, e.g., mimics the cell surface molecule of interest.

Following enrichment of knottin libraries for binders against the cell surface molecule of interest, the yeast plasmids are recovered and sequenced. Additional rounds of FACS can be performed under increased sorting stringency. The binding affinities or kinetic off-rates of individual yeast-displayed knottin clones may then be measured.

Once knottin peptides having an engineered loop that binds to the cell surface molecule of interest have been identified by surface display (e.g., yeast surface display), the engineered knottins may be produced using a suitable method. The small size of knottins makes them amenable to production by both chemical synthesis and recombinant expression. According to certain embodiments, the knottin peptide may be produced by solid phase peptide synthesis followed by in vitro folding. Chemical synthesis permits facile incorporation of unnatural amino acids or other chemical handles into knottin peptides.

Knottin peptides not fused to large heterologous domains are readily synthesized using solid phase peptide chemistry on an automated synthesizer. For example, standard 9-fluorenylmethyloxycarbonyl (Fmoc)-based solid phase peptide chemistry may be employed. The linear peptide may then be folded under conditions that promote oxidation of cysteine side chain thiols to form disulfide bonds, followed by purification, e.g., by reversed-phase high-performance liquid chromatography (RP-HPLC).

In certain aspects, the knottin peptide or a fusion protein that includes the knottin peptide is produced using a recombinant DNA approach. Any suitable strategy for producing the knottin peptide using recombinant methods in a variety of host cell types may be employed. For example, functional knottins have been produced with barnase as a genetic fusion partner, which promotes folding in the E. coli periplasmic space and serves as a useful purification handle. According to certain embodiments, the engineered knottin peptide is expressed in yeast. The yeast strain Pichia pastoris, for example, has been successfully employed to produce 2-10 mg/L of purified engineered knottins. The yeast expression construct may encode one or more tags (e.g., a C-terminal hexahistadine tag for purification by, e.g., metal chelating chromatography (Ni-NTA)). Size exclusion chromatography may then be used to remove aggregates, misfolded multimers, and the like.

Aspects of the present disclosure include nucleic acids that encode the knottin peptides and fusion proteins employed in the conjugates of the present disclosure. That is, provided are nucleic acids that encode any of the knottin peptides and fusion proteins described herein having an engineered loop that binds to a cell surface molecule of interest. In certain aspects, such a nucleic acid is present in an expression vector. The expression vector includes a promoter operably linked to the nucleic acid encoding the knottin peptide, the promoter being selected based on the type of host cell selected to express the knottin peptide. Also provided are host cells that include any of the knottin peptide-encoding nucleic acids of the present disclosure, as well as any expression vectors including the same.

Methods are available for measuring the affinity of knottins for molecules expressed on the surface of cells (e.g., cancer cells, such as mammalian cancer cells) using direct binding or competition binding assays. In a direct binding assay, an equilibrium binding constant (K_{D}) may be measured using a knottin conjugated to a fluorophore or radioisotope, or a knottin that contains an N- or C-terminal epitope tag for detection by a labeled antibody. If labels or tags are not feasible or desired, a competition binding assay can be used to determine the half-maximal inhibitory concentration (IC₅₀), the amount of unlabeled knottin at which 50% of the maximal signal of the labeled competitor is detectable. A K_{D} value can then be calculated from the measured IC₅₀ value. Ligand depletion will be more pronounced when measuring high-affinity interactions over a lower concentration range, and can be avoided or minimized by decreasing the number of cells added in the experiment or by increasing the binding reaction volumes.

In certain aspects, the knottin peptide has an equilibrium binding constant (K_{D}) for the cell surface molecule of from about 0.01 nM to 100 nM, such as from about 0.025 nM to 75 nM, about 0.05 nM to 50 nm, about 0.075 nM to 25 nM, or from about 0.1 nM to 10 nM. In some embodiments, the knottin peptide has an equilibrium binding constant (K_{D}) for the cell surface molecule of from about 0.1 nM to 10 nM. In some embodiments, the knottin peptide has an equilibrium binding constant (K_{D}) for the cell surface molecule of about 0.1 nM. In some embodiments, the knottin peptide has an equilibrium binding constant (K_{D}) for the cell surface molecule of about 0.5 nM. In some embodiments, the knottin peptide has an equilibrium binding constant (K_{D}) for the cell surface molecule of about 1 nM. In some embodiments, the knottin peptide has an equilibrium binding constant (K_{D}) for the cell surface molecule of about 5 nM. In some embodiments, the knottin peptide has an equilibrium binding constant (K_{D}) for the cell surface molecule of about 10 nM.

Detailed guidance and specific protocols for engineering knottins by yeast surface display technology, including knottin library construction and screening, as well as knottin production by chemical synthesis and recombinant expression, and further for cell binding assays to measure the affinity of knottins to molecules (e.g., receptors) expressed on the surface of cells using direct binding or competition binding assays, are described in Moore, S. and Cochran, J. (2012) Engineering Knottins as Novel Binding Agents, Methods in Enzymology, 503, 223-251.

Linkers

The immunostimulant of the present disclosure may be conjugated to the knottin peptide via a variety of suitable linkers. Linkers that find use in the conjugates of the present disclosure include ester linkers, amide linkers, maleimide or maleimide-based linkers; valine-citrulline linkers; hydrazone linkers; N-succinimidyl-4-(2-pyridyldithio)butyrate (SPDB) linkers; Succinimidyl-4-(N-maleimidomethyl)cyclohexane-1-carboxylate (SMCC) linkers; vinylsulfone-based linkers; linkers that include polyethylene glycol (PEG), such as, but not limited to tetraethylene glycol; linkers that include propanoic acid; linkers that include caproleic acid, and linkers including any combination thereof.

In certain aspects, the linker is a chemically-labile linker, such as an acid-cleavable linker that is stable at neutral pH (bloodstream pH 7.3-7.5) but undergoes hydrolysis upon internalization into the mildly acidic endosomes (pH 5.0-6.5) and lysosomes (pH 4.5-5.0) of a target cell (e.g., a cancer cell). Chemically-labile linkers include, but are not limited to, hydrazone-based linkers, oxime-based linkers, carbonate-based linkers, ester-based linkers, etc. According to certain embodiments, the linker is an enzyme-labile linker, such as an enzyme-labile linker that is stable in the bloodstream but undergoes enzymatic cleavage upon internalization into a target cell, e.g., by a lysosomal protease (such as cathepsin or plasmin) in a lysosome of the target cell (e.g., a cancer cell). Enzyme-labile linkers include, but are not limited to, linkers that include peptidic bonds, e.g., dipeptide-based linkers such as valine-citrulline linkers, such as a maleimidocaproyl-valine-citruline-p-aminobenzyl (MC-vc-PAB) linker, a valyl-alanyl-para-aminobenzyloxy (Val-Ala-PAB) linker, and the like. Chemically-labile linkers, enzyme-labile, and non-cleavable linkers are known and described in detail, e.g., in Ducry & Stump (2010) Bioconjugate Chem. 21:5-13.

In certain embodiments, a conjugate of the present disclosure comprises the immunostimulant conjugated to the knottin peptide via a linker described in the Methods of Making Conjugates and Experimental sections hereinbelow, as well as the Figures, of the present disclosure.

Methods of Making Conjugates

Also provided are methods of making conjugates. According to some embodiments, provided are methods of making a knottin-immunostimulant conjugate, the method comprising conjugating an immunostimulant to a knottin peptide via a linker. In certain embodiments, conjugating includes functionalizing the immunostimulant, and conjugating the functionalized immunostimulant to the knottin peptide. According to some embodiments, the knottin peptide and/or the immunostimulant are selected from any of the knottin peptides and/or the immunostimulants described hereinabove and in the Experimental section below.

According to some embodiments, the immunostimulant comprises a primary amine, and functionalizing the immunostimulant comprises reacting the primary amine with an amine-reactive linker. A variety of amine-reactive linkers may be employed when practicing the methods of making the knottin-immunostimulant conjugate. According to one non-limiting example, the amine-reactive linker is an amine-reactive NHS ester linker. In certain embodiments, when an amine-reactive linker is employed, the amine-reactive linker comprises a moiety selected from bicyclo[6.1.0]nonyne (BCN), dibenzocyclooctyne (DBCO), and an azide moiety. When the amine-reactive linker comprises such a moiety, conjugating the functionalized immunostimulant to the knottin peptide may comprise reacting the moiety of the amine-reactive linker with a moiety of the knottin peptide. According to some embodiments, the knottin peptide comprises a non-natural amino acid comprising the moiety of the knottin peptide. For example, the non-natural amino acid may provide an azide moiety. In one non-limiting example, an azide moiety is provided by incorporating one or more 5-azido-L-norvalines into the knottin peptide at the desired location(s). In certain embodiments, the moiety of the knottin peptide with which the functionalized immunostimulant is reacted is an N-terminal amine group.

In some embodiments, a knottin-immunostimulant conjugate of the present disclosure is made according to any of the approaches illustrated in FIGS. 2-4, 8 and 9 and/or as described in the Experimental section below.

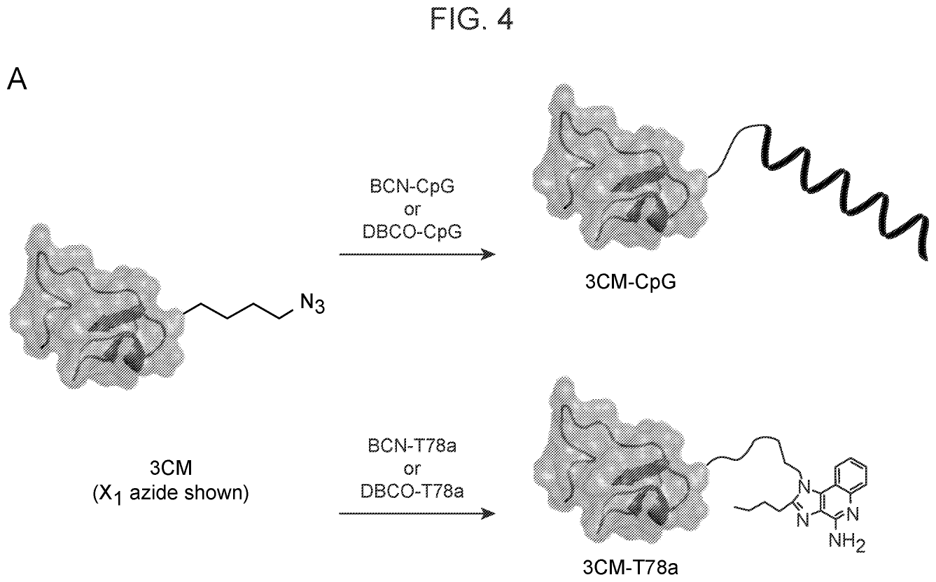

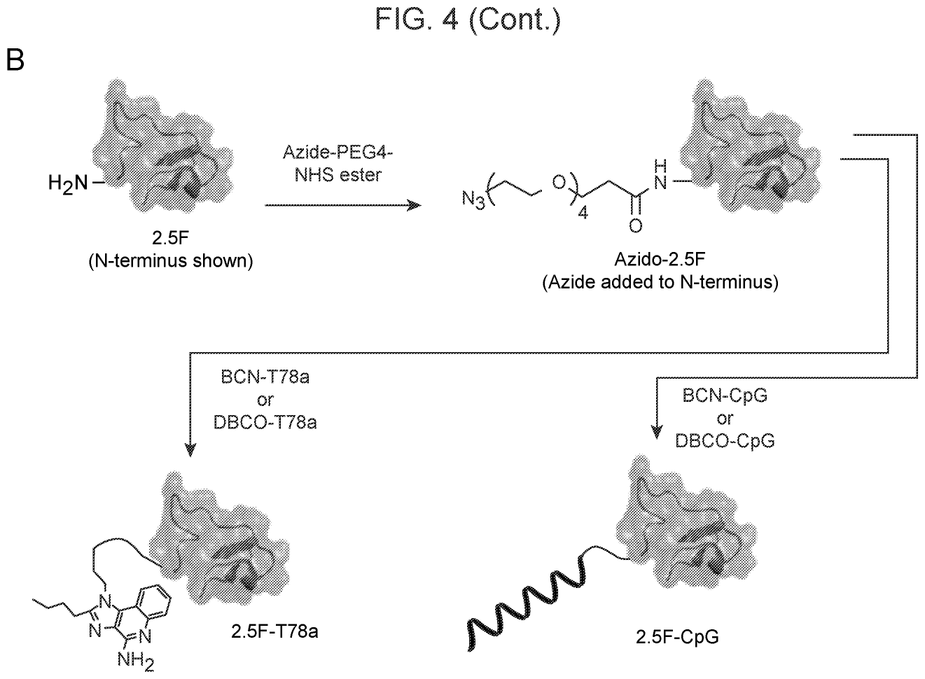

FIG. 2 provides example strategies to functionalize immunostimulants. (A) Immunostimulants with primary amines available for conjugation may be reacted to (B) amine-reactive NHS ester linkers bearing click chemistry handles (e.g. BCN, DBCO, azide), resulting in (C) functionalized immunostimulants bearing click chemistry handles (e.g. BCN, DBCO, azide), which are used to conjugate immunostimulants to tumor-targeting agents (described in FIG. 4).

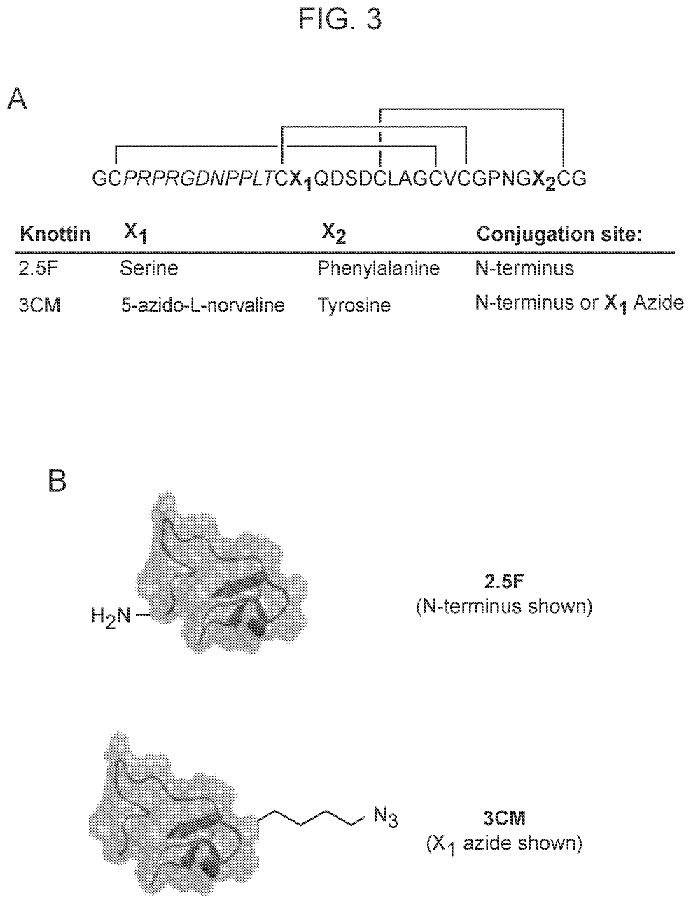

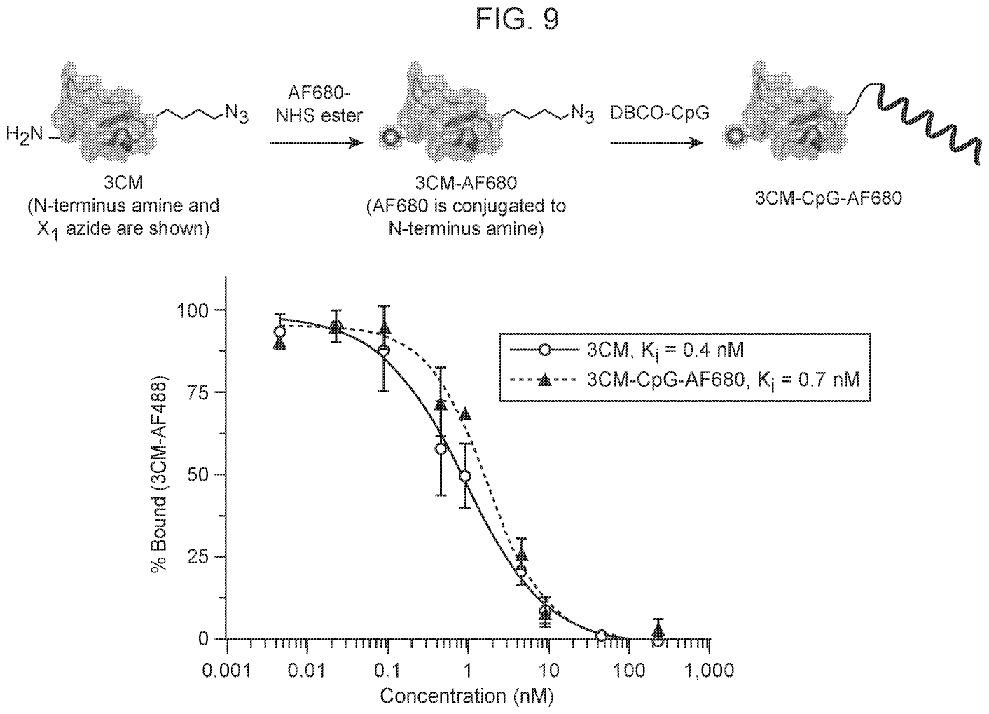

FIG. 3 provides example sequences and illustrations of knottin peptides. (A) The sequences of knottin peptides 2.5F and 3CM with integrin-binding loop (PRPRGDNPPLT) and disulfide linkages of the cysteine-knot scaffold shown. (B) Illustration of knottin peptide structure for 2.5F with N-terminal amine group shown and 3CM with 5-azido-L-norvaline at X₁ position shown. The knottin peptide 2.5F can be conjugated at the N-terminal amine group, whereas 3CM can be conjugated at the X, azide site (unnatural amino acid incorporated for this purpose). 3CM also has an N-terminal amine group available that can be reacted to immunostimulants or probes, such as fluorophores (see FIG. 9). In addition, the phenylalanine at the X₂ position of 2.5F may be substituted for tyrosine in 3CM to facilitate concentration measurements by UV absorption. Either amino acid at X₂ can be used without compromising binding affinity.

Shown in FIG. 4 are example strategies to conjugate knottin peptides to functionalized immunostimulants. (A) The X, azide of 3CM may be reacted to BCN or DBCO-functionalized immunostimulants (e.g., an oligonucleotide-based TLR 9 agonist comprising one or more CpG dinucleotides (e.g., a CpG ODN), sometimes referred to herein in the figures, descriptions thereof, and Experimental section as “CpG”; or T78a) using strain promoted azide-alkyne cycloaddition (SPAAC). (B) The N-terminal amine of 2.5F may be modified using an azide-PEG4-NHS ester linker to incorporate an N-terminal azide. The N-terminal azide may be reacted to BCN or DBCO-functionalized immunostimulants using SPAAC.

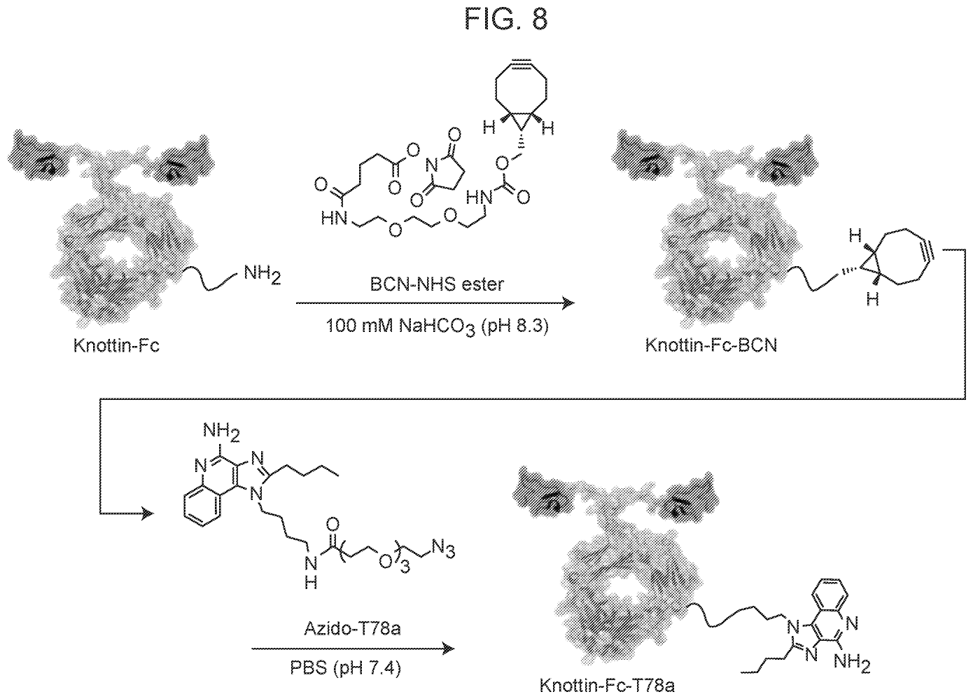

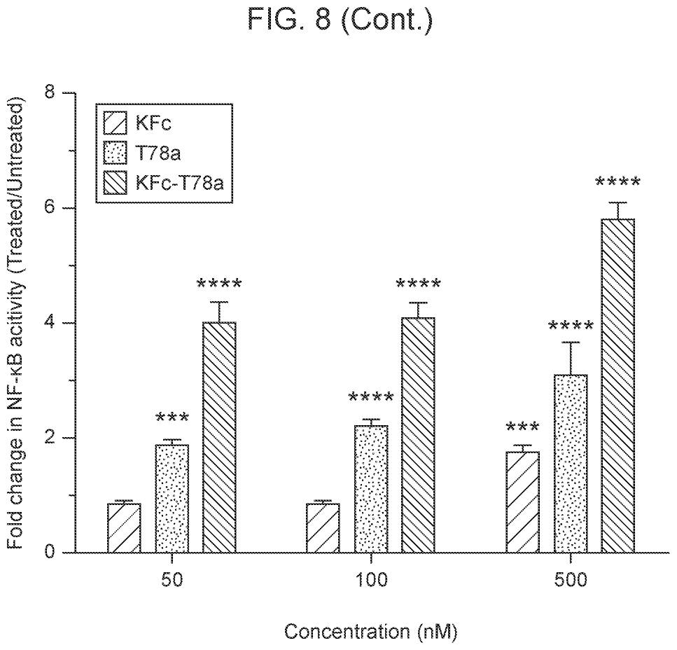

FIG. 8 (top) schematically illustrates a method of making a knottin-immunostimulant conjugate (knottin-Fc-T78a in this example) according to embodiments of the present disclosure. As shown, to conjugate knottin-Fc (KFc) to T78a to produce KFc-T78a, BCN-modified KFc and Azido-T78a were employed.

FIG. 9 (top) schematically illustrates a method of making a knottin-immunostimulant conjugate, where the knottin is further conjugated to a detectable label. In this example, the detectable label is AlexaFluor 680. According to this example approach to synthesize 3CM-CpG-AF680, 3CM was modified at the N-terminus with AF680-NHS ester (fluorophore) and modified at the X, azide with DBCO-CpG.

### Compositions

As summarized above, the present disclosure provides compositions. The compositions may include any of the conjugates of the present disclosure, including any of the conjugates described in the Conjugates section above, which is incorporated but not reiterated herein for purposes of brevity.

In certain aspects, the compositions include a conjugate of the present disclosure present in a liquid medium. The liquid medium may be an aqueous liquid medium, such as water, a buffered solution, and the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a protease inhibitor, glycerol, and the like may be present in such compositions.

Pharmaceutical compositions are also provided. The pharmaceutical compositions include any of the conjugates of the present disclosure, and a pharmaceutically-acceptable carrier. The pharmaceutical compositions generally include a therapeutically effective amount of the conjugate. By “therapeutically effective amount” is meant a dosage sufficient to produce a desired result, e.g., an amount sufficient to effect beneficial or desired therapeutic (including preventative) results, such as a reduction in cellular proliferation in an individual having a cell proliferative disorder (e.g., cancer) associated with the cell surface molecule to which the engineered loop binds, etc. An effective amount may be administered in one or more administrations.

A conjugate of the present disclosure can be incorporated into a variety of formulations for therapeutic administration. More particularly, the conjugate can be formulated into pharmaceutical compositions by combination with appropriate pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the conjugates of the present disclosure suitable for administration to an individual (e.g., suitable for human administration) are generally sterile and may further be free of detectable pyrogens or other contaminants contraindicated for administration to an individual according to a selected route of administration.

In pharmaceutical dosage forms, the conjugate can be administered alone or in appropriate association, as well as in combination, with other pharmaceutically-active compounds. The following methods and excipients are merely examples and are in no way limiting.

For oral preparations, the conjugate can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The conjugates can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The pharmaceutical composition may be in a liquid form, a lyophilized form or a liquid form reconstituted from a lyophilized form, where the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the conjugate may be prepared in a pH-buffered solution, e.g., at pH ranging from about 4.0 to about 8.0, such as from about 4.5 to about 7.5, e.g., from about 5.0 to about 7.0. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

### Kits

Aspects of the present disclosure further include kits. In some embodiments, a subject kit includes any of the conjugates of the present disclosure (including any of the conjugates described in the Conjugates section above, which is incorporated but not reiterated herein for purposes of brevity) or a pharmaceutical composition comprising same, and instructions for administering the pharmaceutical composition to an individual in need thereof. In certain embodiments, the conjugate comprises a knottin peptide comprising an engineered loop that binds to a cancer cell surface molecule and/or molecule on the surface of tumor vasculature cells (e.g., an engineered loop that binds to any of the tumor antigens, cell adhesion receptors (e.g., integrins), etc. described elsewhere herein), and the instructions are for administering the pharmaceutical composition to an individual having cancer to treat the cancer.

In some embodiments, the conjugate or pharmaceutical composition is present in one or more (e.g., two or more) unit dosages. The term “unit dosage”, as used herein, refers to physically discrete units suitable as unitary dosages for human and animal subjects, each unit containing a predetermined quantity of the conjugate or composition calculated in an amount sufficient to produce the desired effect. The amount of the unit dosage depends on various factors, such as the particular conjugate employed, the effect to be achieved, and the pharmacodynamics associated with the conjugate, in the individual. In yet other embodiments, the kits may include a single multi dosage amount of the conjugate or pharmaceutical composition.

Components of the kits may be present in separate containers, or multiple components may be present in a single container.

The instructions included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

### Methods of Use

As summarized above, also provides are methods of using the conjugates of the present disclosure. In some embodiments, the methods including using any of the conjugates described in the Conjugates section above, which is incorporated but not reiterated herein for purposes of brevity.

In some embodiments, provided are methods that include administering to an individual in need thereof a therapeutically effective amount of any of the conjugates or any of the pharmaceutical compositions of the present disclosure. In certain embodiments, the individual has cancer, the knottin peptide of the conjugate comprises an engineered loop that binds to a cell surface molecule on cancer cells and/or tumor vasculature cells present in the individual, and a pharmaceutical composition comprising the conjugate is administered to the individual in an amount effective to treat the cancer. Accordingly, aspects of the present disclosure include methods of treating cancer by administering to an individual having cancer a therapeutically effective amount of any of the conjugates or any of the pharmaceutical compositions of the present disclosure.

A variety of individuals are treatable according to the subject methods. Generally such individuals are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the individual is a human. In some embodiments, the individual is an animal model, such as a mouse model.

In some embodiments, an effective amount of the conjugate (or pharmaceutical composition comprising same) is an amount that, when administered alone (e.g., in monotherapy) or in combination (e.g., in combination therapy) with one or more additional therapeutic agents, in one or more doses, is effective to reduce the symptoms of a medical condition of the individual (e.g., cancer, etc.) by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the symptoms in the individual in the absence of treatment with the conjugate or pharmaceutical composition.

In some embodiments the individual has cancer, and the methods of the present disclosure find use in treating the individual's cancer. In some embodiments, the individual has a cancer characterized by the presence of a solid tumor, a semi-solid tumor, a primary tumor, a metastatic tumor, a liquid tumor (e.g., a leukemia, a lymphoma, or the like), and/or the like. In some embodiments, the individual has a cancer selected from breast cancer, glioblastoma, neuroblastoma, head and neck cancer, gastric cancer, ovarian cancer, skin cancer (e.g., basal cell carcinoma, melanoma, or the like), lung cancer, colorectal cancer, prostate cancer, glioma, bladder cancer, endometrial cancer, kidney cancer, leukemia (e.g., acute myeloid leukemia (AML)) liver cancer (e.g., hepatocellular carcinoma (HCC), such as primary or recurrent HCC), non-Hodgkin lymphoma, pancreatic cancer, thyroid cancer, a B-cell malignancy, any combinations thereof, and any sub-types thereof. According to certain embodiments, the individual has a condition characterized by the presence of neoplastic and/or malignant cells.

By “treat”, “treating” or “treatment” is meant at least an amelioration of the symptoms associated with the medical condition (e.g., cell proliferative disorder, e.g., cancer) of the individual, where amelioration is used in a broad sense to refer to at least a reduction in the magnitude of a parameter, e.g. symptom, associated with the medical condition being treated. As such, treatment also includes situations where the medical condition (e.g., cancer), or at least symptoms associated therewith, are completely inhibited, e.g., prevented from happening, or stopped, e.g., terminated, such that the individual no longer suffers from the medical condition, or at least the symptoms that characterize the medical condition.

The conjugate or pharmaceutical composition may be administered to the individual using any available method and route suitable for drug delivery, including in vivo and ex vivo methods, as well as systemic and localized routes of administration. Conventional and pharmaceutically acceptable routes of administration include intranasal, intramuscular, intra-tracheal, subcutaneous, intradermal, topical application, ocular, intravenous, intra-arterial, nasal, oral, and other enteral and parenteral routes of administration. In some embodiments, the administering is by parenteral administration. Routes of administration may be combined, if desired, or adjusted depending upon the conjugate and/or the desired effect. The conjugates or pharmaceutical compositions may be administered in a single dose or in multiple doses. In some embodiments, the conjugate or pharmaceutical composition is administered intravenously. In some embodiments, the conjugate or pharmaceutical composition is administered by injection, e.g., for systemic delivery (e.g., intravenous infusion) or to a local site, e.g., intratumoral injection, peritumoral injection, and/or the like.

In some embodiments, the individual has a solid tumor. In some embodiments, when the individual has a solid tumor, the methods include administering a knottin-immunostimulant conjugate of the present disclosure to the individual. As demonstrated herein, such conjugates exhibit the unexpected capability of localizing to solid tumors following systemic administration and achieve substantially greater therapeutic efficacy than the corresponding non-conjugated immunostimulant. According to some embodiments, the individual has a solid tumor, the administering is by systemic administration, and the immune cell microenvironment of the solid tumor is characterized by one or any combination of increased percentage of CD8+ T cells, increased percentage of CD4+ T cells, increased percentage of B cells, and/or decreased percentage of myeloid-derived suppressor cells (MDSCs), as compared to the immune cell microenvironment of the tumor when the immunostimulant alone is administered systemically to the individual. According to some embodiments, the individual has a solid tumor, the administering is by systemic administration, and the immune cell microenvironment of the solid tumor as assessed by one or any combination of the percentage of CD8+ T cells, the percentage of CD4+ T cells, the percentage of B cells, and/or the percentage of myeloid-derived suppressor cells (MDSCs), is not statistically significantly different as compared to the immune cell microenvironment of the tumor when the immunostimulant alone is administered intratumorally to the individual.

In some embodiments, the individual has a cancer the treatment of which requires the conjugate to cross the blood-brain barrier (BBB). A non-limiting example of such a cancer is a brain tumor, e.g., glioblastoma, or the like. In some embodiments, when the individual has a cancer the treatment of which requires the conjugate to cross the BBB, the methods include administering a low molecular weight conjugate to the individual, such as a knottin-immunostimulant conjugate of the present disclosure.

The following examples are offered by way of illustration and not by way of limitation.

### EXPERIMENTAL

### Example 1—Knottin-CpG Conjugates with CpG Incorporated at Different Sites

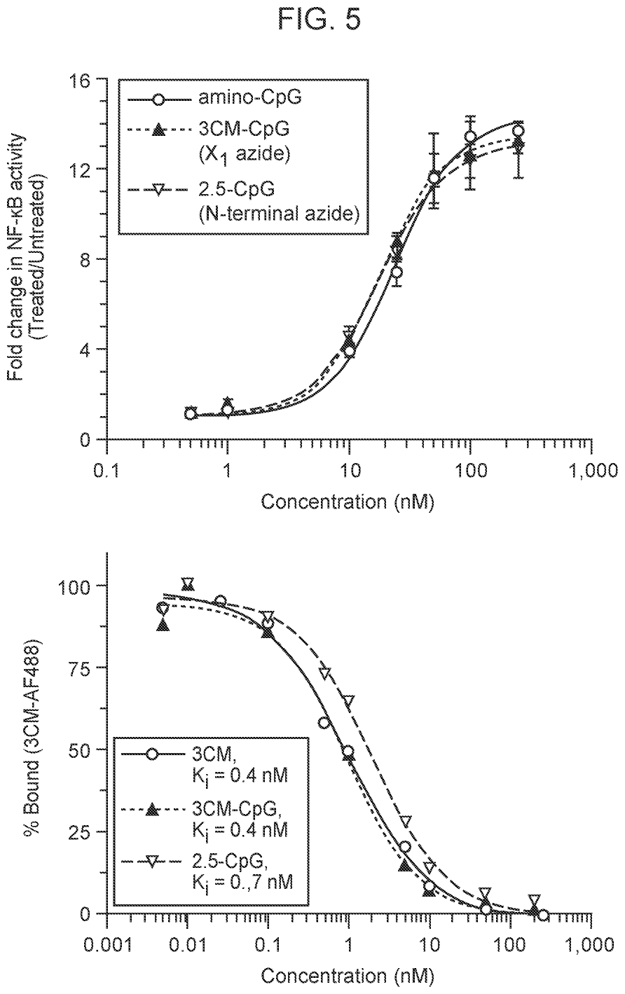

In this example, knottin peptide-immunostimulant conjugates were prepared with the immunostimulant incorporated at different sites. This particular example involves the knottin peptides 2.5F and 3CM conjugated to a CpG ODN (referred to herein and in the figures as “CpG”) incorporated at different sites. Shown in the left panel of FIG. 5 are RAW-Blue NF-κB activation assay results for amino-CpG, 3CM-CpG (X, azide), and 2.5F-CpG (N-terminal azide). BCN-CpG was conjugated at sites shown in parentheses. Shown in the right panel of FIG. 5 are competition binding assay results comparing 3CM, 3CM-CpG (X, azide), and 2.5F-CpG (N-terminal azide).

The knottin-CpG conjugates (at either conjugation site) exhibited similar NF-κB activation profiles compared to amino-CpG (positive control) and also exhibited similar binding profiles compared to unmodified 3CM (positive control). Thus, either conjugation site (X, azide or N-terminal azide) can be used to synthesize knottin-CpG conjugates without negatively impacting TLR agonist activity or binding affinity. Amino-CpG exhibited the same NF-κB activation profile as unmodified CpG (data not shown).

### Example 2—Knottin-CpG Conjugates Synthesized with Different Linkers

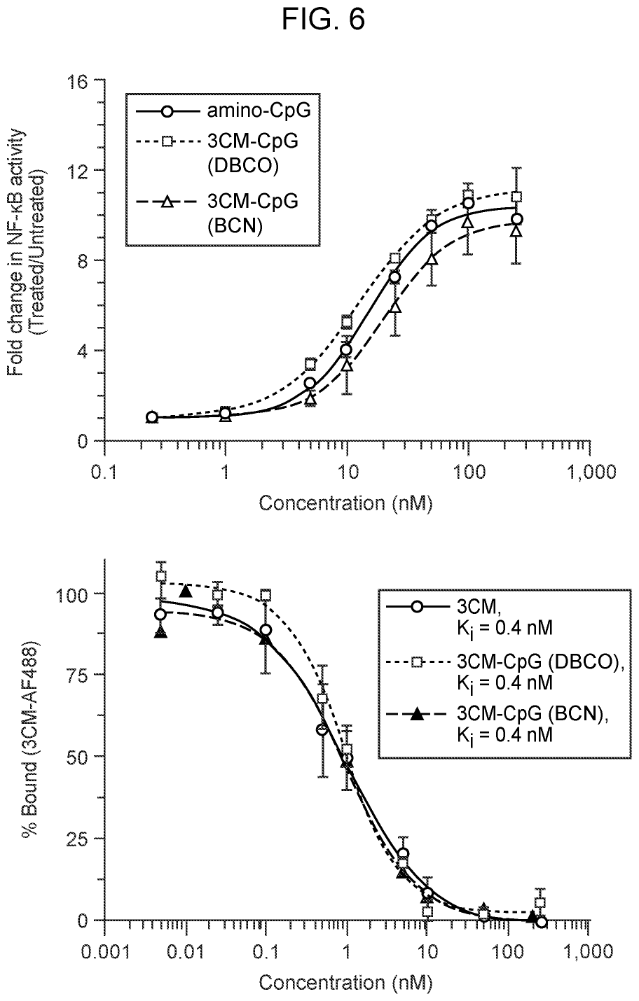

In this example, knottin peptide-immunostimulant conjugates were prepared using different linkers. This particular example involves the knottin peptide 3CM conjugated to CpG using different linkers. Shown in the left panel of FIG. 6 are RAW-Blue NF-κB activation assay results for amino-CpG, 3CM-CpG (DBCO), and 3CM-CpG (BCN). The CpG was conjugated to the X₁ azide on 3CM for both conjugates. Shown in the right panel of FIG. 6 are competition binding assay results comparing 3CM, 3CM-CpG (DBCO), and 3CM-CpG (BCN).

The knottin-CpG conjugates (with either linker) exhibited similar NF-κB activation profiles compared to amino-CpG (positive control) and also exhibited similar binding profiles compared to unmodified 3CM (positive control). Thus, either linker strategy (DBCO or BCN) can be used to synthesize knottin-CpG conjugates without negatively impacting TLR agonist activity or binding affinity.

### Example 3—Knottin-TLR7/8 Agonist (T78a) Conjugate

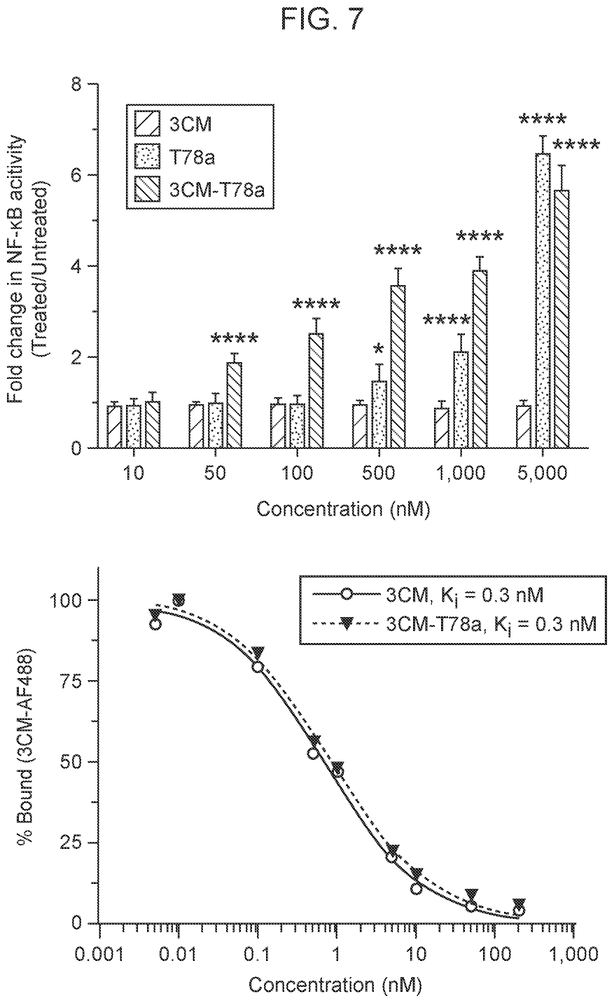

In this example, a knottin peptide-immunostimulant was prepared and tested, where the knottin peptide was 3CM and the immunostimulant was the TLR7/8 agonist T78a. Shown in the left panel of FIG. 7 are RAW-Blue NF-κB activation assay results for 3CM, T78a, and 3CM-T78a. Shown in the right panel of FIG. 7 are competition binding assay results comparing 3CM and 3CM-T78a.

The TLR7/8 agonist (T78a) activated NF-κB only at the higher concentrations tested (500-5,000 nM), whereas the 3CM-T78a conjugate induced activation at lower concentrations (50-5,000 nM). In addition, 3CM-T78a exhibited a similar binding profile as unconjugated 3CM and thus retains high affinity to integrins.

### Example 4—Knottin-Fc Immunostimulant Conjugates

In this example, conjugates comprising a knottin peptide fused to an Fc domain and conjugated to an immunostimulant were synthesized and tested. FIG. 8 (top) schematically illustrates the method of making the conjugate (knottin-Fc-T78a in this example). As shown, to conjugate knottin-Fc (KFc) to T78a to produce KFc-T78a, BCN-modified KFc and Azido-T78a were employed. FIG. 8 (bottom panel) shows RAW-Blue NF-κB activation assay results for KFc, T78a, and KFc-T78a conjugate.

NF-κB activation of KFc-T78a is significantly higher than KFc or T78a at each concentration tested (p<0.0001). For synthesis, the BCN and azide groups may be switched such that KFc is modified with azido-NHS ester linker and conjugated to BCN-T78a (or BCN-CpG). DBCO can be substituted for BCN as well.

### Example 5—Detectably Labeled Knottin-Immunostimulant Conjugates

In this example, conjugates comprising a knottin peptide conjugated to an immunostimulant and a detectable label (here, AlexaFluor 680) were synthesized and tested. FIG. 9 (top) schematically illustrates the approach employed in this example. To synthesize 3CM-CpG-AF680, 3CM was modified at the N-terminus with AF680-NHS ester (fluorophore) and modified at the X, azide with DBCO-CpG. FIG. 9 (bottom) shows competition binding assay results for 3CM-CpG-AF680 compared to 3CM.

The binding affinity of 3CM-CpG-AF680 is not significantly different than 3CM (determined by an unpaired Student's t test). Data represent the mean (±standard deviation, SD) of three independent experiments.

### Example 6—Non-Invasive In Vivo and Ex Vivo Fluorescence Imaging

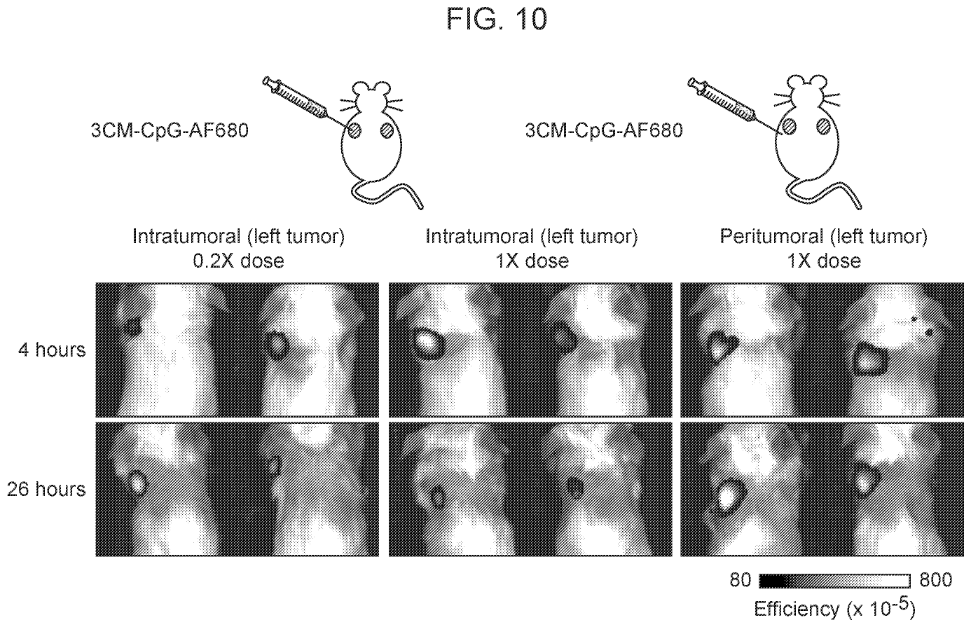

In this example, non-invasive in vivo fluorescence imaging of a 3CM-CpG-AF680 conjugate injected intratumorally and peritumorally was performed. Results are shown in FIG. 10. Mice bearing two CT26 colon carcinoma tumors on left and right shoulders were injected intratumorally (IT; in left tumor) or peritumorally (PT; next to left tumor) with 3CM-CpG-AF680 at the indicated doses. “Dose” is the typical dose (equal molar) for intratumoral CpG therapy=5.2 nmoles (50 μg). Time shown in hours to the left of images is hours post-injection. The 3CM-CpG-AF680 conjugate did not localize to non-injected tumor sites following IT or PT injection after 4h or 26h.

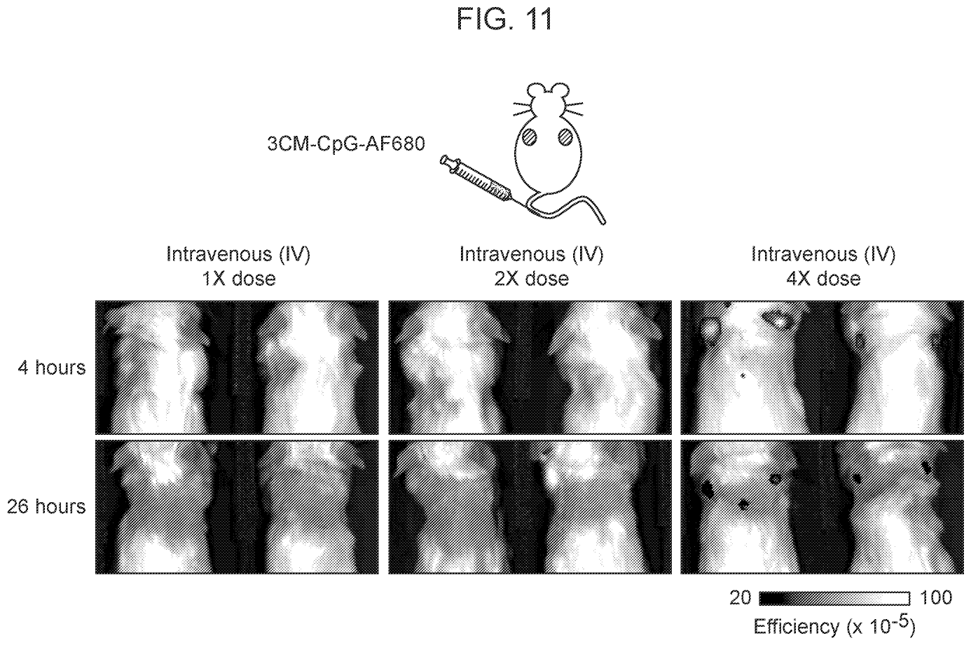

Also in this example, non-invasive in vivo fluorescence imaging of a 3CM-CpG-AF680 conjugate injected intravenously was performed. Results are shown in FIG. 11. Mice bearing two CT26 colon carcinoma tumors on left and right shoulders were injected intravenously (IV; tail vein) with 3CM-CpG-AF680 at the indicated doses. “Dose” is the typical dose (equal molar) for intratumoral CpG therapy=5.2 nmoles (50 μg). Time shown in hours to the left of images is hours post-injection. 3CM-CpG-AF680 (4× dose) localized to both tumors within 4 hours after IV injection and is retained in tumor site for over 24h. Lower doses may also result in tumor localization, but is not measurable by in vivo fluorescence imaging.

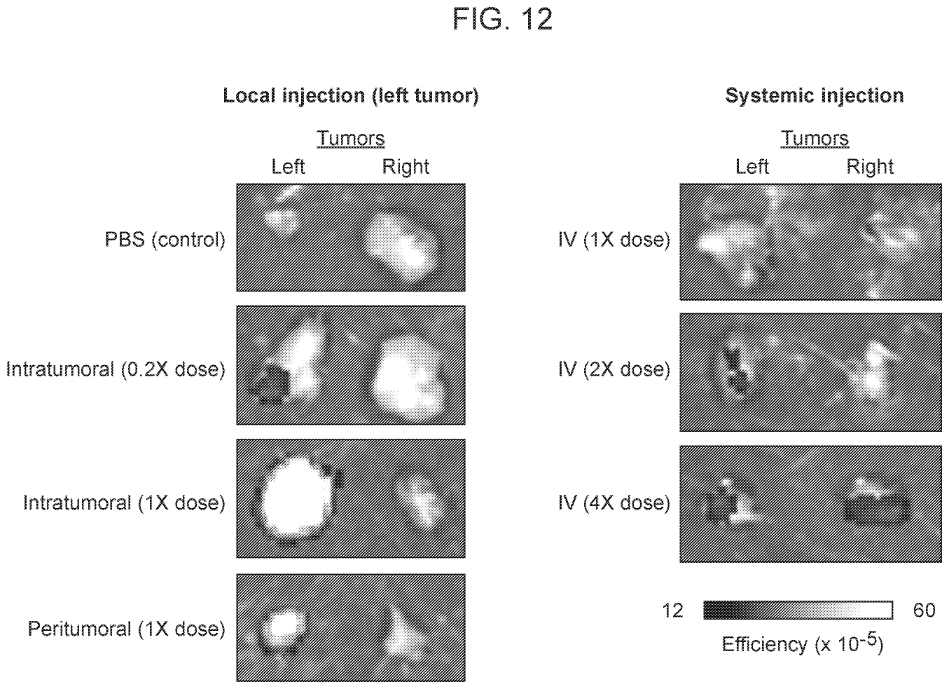

Tumors were excised after 26h and tumor fluorescence ex vivo from mice injected with 2× dose and 4× dose (see FIG. 12) was observed. Results are shown in FIG. 12. Mice bearing two CT26 colon carcinoma tumors on left and right shoulders were injected intratumorally (IT; in left tumor), peritumorally (PT; next to left tumor), or intravenously (IV; tail vein) with 3CM-CpG-AF680 at indicated doses. “Dose” is the typical dose (equal molar) for intratumoral CpG therapy=5.2 nmoles (50 ug). Tumors were excised for imaging at 26h post-injection. Ex vivo images support observations from in vivo imaging. A) 3CM-CpG-AF680 does not localize to non-injected tumor (right tumor) following IT or PT injection after 26h.

B) 3CM-CpG-AF680 delivered PT results in tumor uptake at 26h post-injection; conjugate may also be present at peritumoral injection site (in area surrounding tumor). C) 3CM-CpG-AF680 (4× dose) delivered IV localizes to both tumors at 26h post-injection. Localization was also observed in one tumor with 3CM-CpG-AF680 (2× dose) delivered IV.

### Example 7—Therapeutic Efficacy of Knottin-Immunostimulant Conjugates as Indicated by Tumor Growth

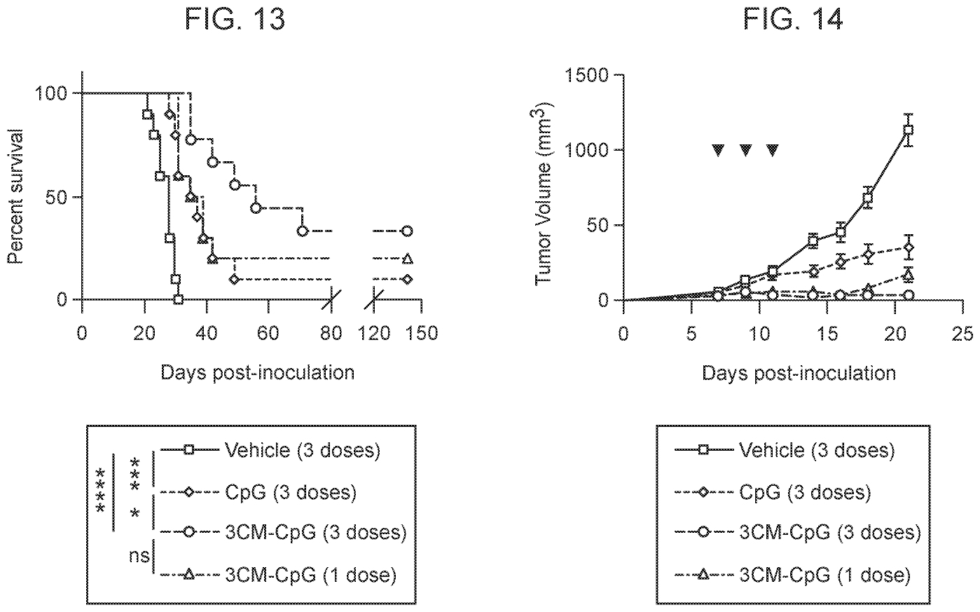

In this example, the therapeutic efficacy of an example knottin-immunostimulant conjugate was assessed. As proof of principle, a 3CM-CpG conjugate was employed in this example. The aggressive 4T1 breast cancer mouse model was used. Survival curves demonstrating therapeutic efficacy of 3CM-CpG in 4T1-Luc breast carcinoma (n=9-10) are shown in FIG. 13. Mice with established tumors that received three doses of intravenous knottin-CpG had significantly prolonged survival compared to mice treated with three doses of intravenous unmodified CpG or vehicle control.

Furthermore, knottin-CpG treatment induced complete tumor regression in 6 of 9 mice with the remaining 3 mice showing delayed tumor growth compared to vehicle-treated mice. For the mice that exhibited complete tumor regression, 50% of the mice were cured (3 of 6 mice) with no signs of tumor recurrence for several months following tumor regression. These results are significant and unexpected, as it has been unprecedented to achieve cures in the 4T1 model with alternate systemic monotherapies.

Also performed was a rechallenge experiment where, at 141 days post-inoculation, the surviving mice were reinjected with 4T1-luc tumor cells subcutaneously in the opposite side of the abdomen using twice the number of cells used for the original inoculation. Naïve mice were also injected with the same number of tumor cells. At 16 days post-inoculation, none of the surviving mice had tumors, whereas all of the naïve mice had established tumors.

Shown in FIG. 14 is 4T1-Luc average tumor growth over time. Average tumor volume is plotted for each group up until the first mouse was euthanized (from vehicle group). Arrows indicate treatment days (7, 9, 11) for groups receiving 3 doses; only day 7 for 1 dose group. Data represent mean±SEM (n=9-10).

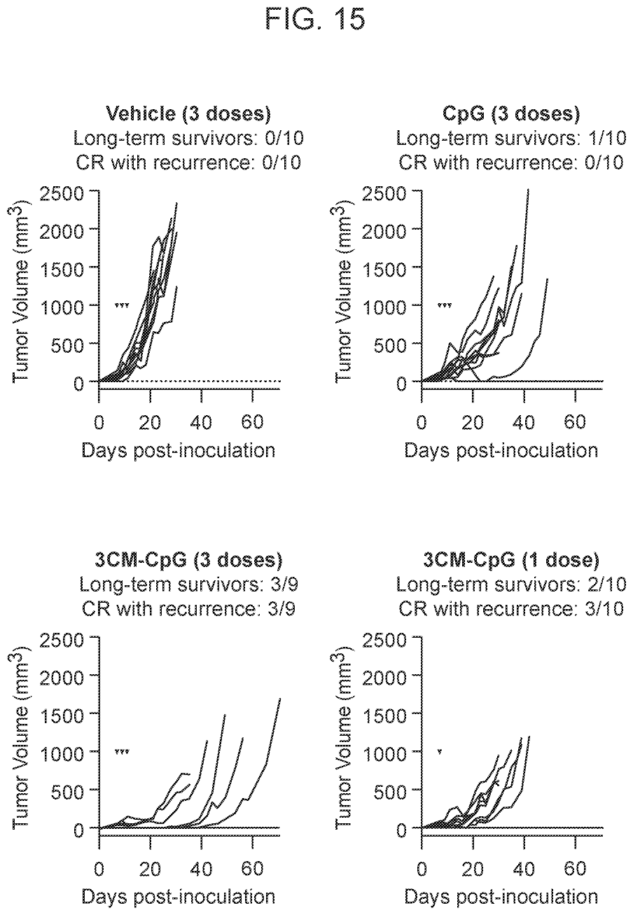

Shown in FIG. 15 are individual 4T1-Luc tumor growth curves. The fraction of mice from each group demonstrating a complete response (CR) without recurrence (“long-term survivors”) and the fraction of mice demonstrating CR with recurrence is shown on individual plots. CR is defined as complete regression of tumor. CR with recurrence is defined as complete regression of tumor followed by regrowth at some point after tumor regression.

### Example 8—Transformation of the Tumor Immune Microenvironment by Knottin-Immunostimulant Conjugates

In this example, the mechanism of therapeutic efficacy of an example knottin-immunostimulant conjugate administered intravenously was assessed by assaying for tumor-infiltrating immune cells. One motivation behind developing knottin-immunostimulant conjugates was to enable systemic injection with targeted delivery to tumor sites. If the knottin-immunostimulant reaches the tumor site and stimulates an immune response, it is expected that the immune cell profile in the tumor will change to facilitate this anti-tumor immune response (e.g., increased CD8+ T cells). As proof of principle, a 3CM-CpG conjugate was employed in this example. As a positive control for the desired shift in the immune cell profile, included was a group of mice treated with intratumoral CpG directly injected into the tumor (thus reaching the tumor site) and is known to stimulate an immune response locally.

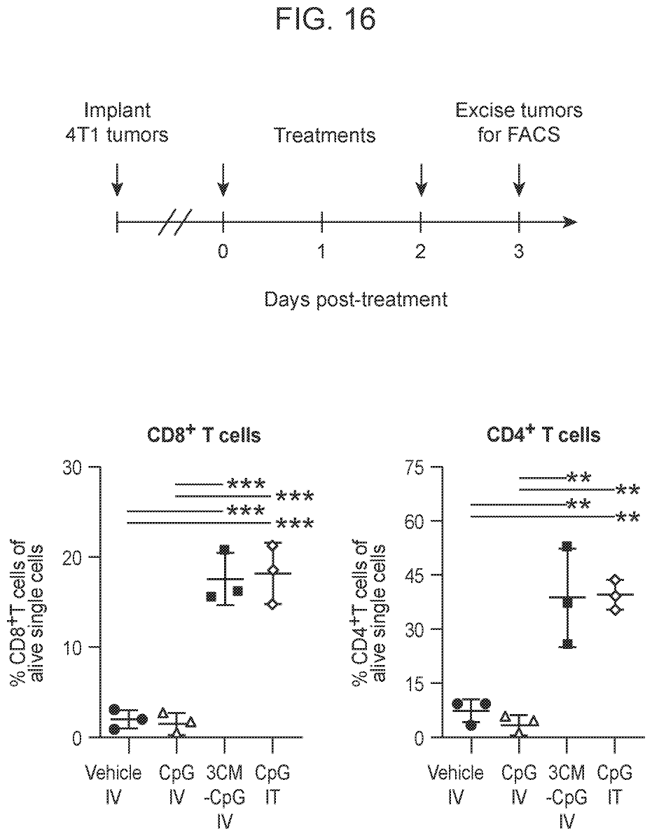

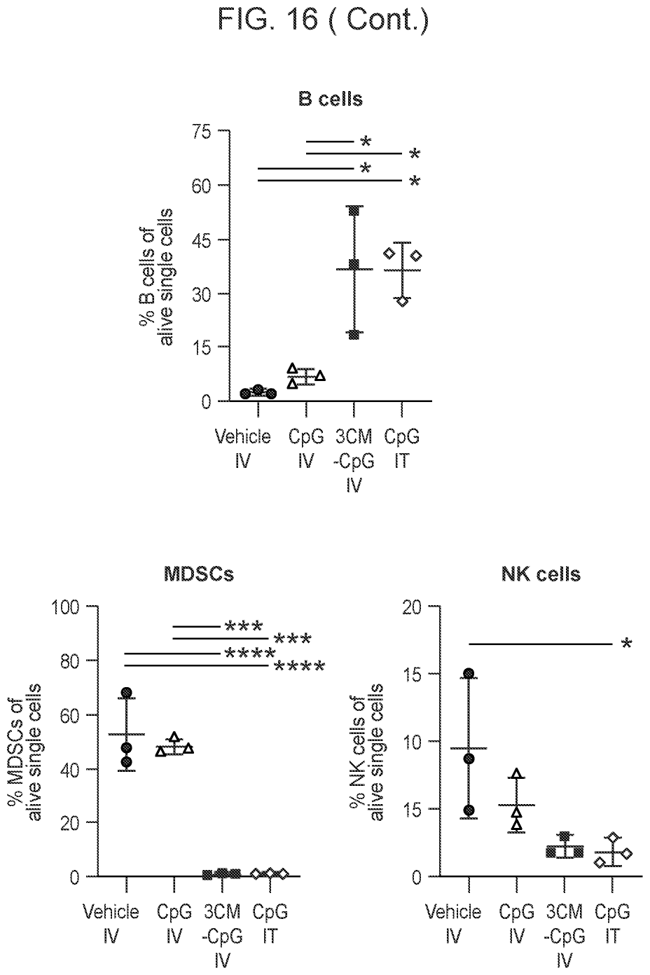

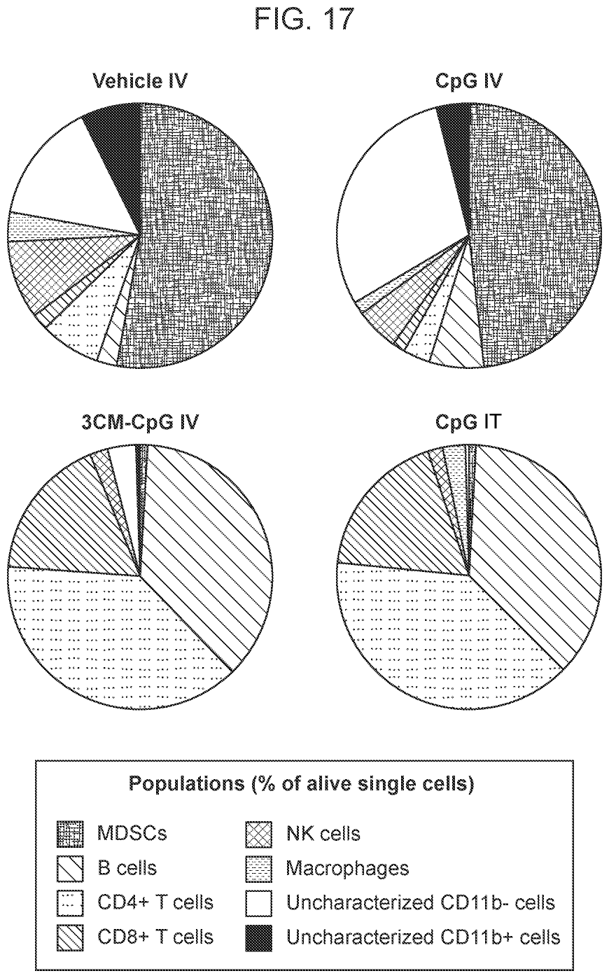

4T1-luc cells were implanted subcutaneously in one side of the abdomen of BALB/c mice and allowed to grow for 9 days. Once tumors had developed, mice were treated twice according to the schematic (FIG. 16, top left) with intravenous (IV) or intratumoral (IT) injections for the following groups (n=3 mice per group): Vehicle IV, CpG IV (18.2 nmol), 3CM-CpG IV (18.2 nmol), and CpG IT (5.2 nmol; typical IT dose). At 3 days post-treatment, tumors were excised and analyzed for tumor-infiltrating immune cells via FACS. Shown in FIG. 16 are plots of the abundance of different immune populations (as % of total alive single cells), including CD8+ T cells, CD4+ T cells, B cells, myeloid-derived suppressor cells (MDSCs), and NK cells. Statistical analyses were performed using one-way ANOVA with Tukey's multiple comparisons test. Each group was compared to every other group. Group comparisons that were statistically significant are marked with a black line drawn between the two groups with asterisks to the right: *P<0.05, **P<0.01, ***P<0.001, ****P<0.0001. All other unmarked group comparisons were not significantly different. Shown in FIG. 17 are pie charts summarizing the average abundance (% total alive single cells) of immune cell populations and two uncharacterized cell populations (“Other”) for each treatment group. Uncharacterized cells, which may include tumor, stromal, and other immune cells not defined in this analysis, were divided into two populations to indicate their CD11b expression.

These results demonstrate that 3CM-CpG IV significantly transforms the tumor immune landscape compared to Vehicle IV and CpG IV treatments as indicated by an increased percentage of CD8+ T cells, CD4+ T cells, and B cells, as well as a decrease in myeloid-derived suppressor cells (MDSCs). Furthermore, this dramatic shift in immune cell profile with 3CM-CpG IV was indistinguishable from the shift observed from treatment with CpG IT. These results demonstrate that systemic injection of knottin-immunostimulant conjugate can induce the immune cell populations to shift as if the immunostimulant were directly injected into that tumor site.

### Materials and Methods

Cell Lines and Mice

B16F10 melanoma and CT26 colon carcinoma lines were obtained from ATCC, and 4T1-Luc breast carcinoma cell line were obtained as a gift. Tumor cells were cultured in complete medium (RPMI 1640 with 50 μM 2-mercaptoethanol for 4T1-Luc and CT26; DMEM for B16-F10) containing 10% fetal bovine serum and 1% penicillin/streptomycin. Cell lines were routinely tested for mycoplasma contamination. Six- to 8-week-old female BALB/c mice were purchased from Charles River Laboratory.

NF-κB Activation Assays

Agonist potency was evaluated using the RAW-Blue reporter cell line (Invivogen), derived from murine RA W264.7 macrophages. Stimulation of the reporter cell line with TLR agonists induces signaling pathways leading to the activation of NF-κB and AP-1, and the subsequent production of secreted embryonic alkaline phosphatase (SEAP). RAW-Blue cells were incubated with TLR agonists (free or conjugated) at various concentrations for 24h. The levels of SEAP in the supernatant were quantified by colorimetry using QUANTI-Blue detection medium following the manufacturer's protocol (Invivogen). Data are reported as fold change in NF-κB activity compared to the untreated control. Error bars represent the standard deviation of experiments performed in triplicate. Statistical differences between treatment conditions and untreated controls were determined by ordinary two-way ANOVA analysis and Tukey's multiple comparisons test using Prism software (GraphPad). For statistical significance, *p≤0.05, **p≤0.01, ***p≤0.001, and ****p≤0.0001, compared to the untreated control.

Competition Binding Assay

To compare the relative binding affinities of unlabeled knottin and knottin-TLR agonist conjugates, cell-based competition binding assays were performed as previously described in Cox et al. (2016) Angew Chem Int Ed. 55(34): 9894-7 with some modifications. Alexa Fluor 488-labeled 3CM (3CM-AF488) was used as a competitor to compare the binding affinity of unlabeled ligands (i.e. non-fluorescent 3CM-immunostimulant conjugates).

B16F10 melanoma cells (5×10⁴ per sample) were detached with cell dissociation buffer, washed with PBS, and incubated with 0.5 nM 3CM-AF488 and varying concentrations of unlabeled ligands in 200 μL of integrin-binding buffer (IBB: 25 mM Tris pH 7.4, 150 mM NaCl, 2 mM CaCl₂, 1 mM MgCl₂, 1 mM MnCl₂, and 0.1% BSA) at 4° C. for 3 hours. For binding assays involving CpG, cells were pretreated with unmodified CpG prior to incubation with integrin-binders to reduce ligand depletion mediated through cell surface DNA-binding interactions. Cells were pretreated for 10 min at room temperature with 500 nM unmodified CpG (100 μL/sample) before adding to solutions of competitor and unlabeled ligands (final volume 200 μL/sample).

The cell-bound fluorescence remaining after washing several times with PBS+0.5% BSA was analyzed by flow cytometry. The geometric mean fluorescence intensity (MFI) from each sample was determined, and the dataset was normalized such that the MFI from cells treated with competitor alone was equal to 100% Bound. The half-maximal inhibitory concentration (IC50) values were determined by nonlinear regression analysis for conversion to equilibrium dissociation constant (Ki) values using the Cheng-Prusoff relationship.

In Vivo Fluorescence Imaging

CT26 colon cancer cells (5×10⁵) were injected subcutaneously on both the left and right shoulders. After 5 days when both tumors were established, mice were injected either intratumorally (IT; in left tumor), peritumorally (PT; next to left tumor), or intravenously (IV) via the tail vein with 3CM-CpG-AF680 at indicated doses. “Dose” is the equal molar amount of the standard intratumoral dose for CpG (5.2 nmol, 50 ug). Thus, 0.2× dose=1 nmol, 1× dose=5.2 nmol, 2× dose=10.4 nmol, and 4× dose=20.8 nmol.

At 4h and 26h post-injection, mice were anesthetized using 2% isoflurane gas and imaged using a Spectral Instruments Imaging Ami Imager. After the last scan at 26h post-injection, one mouse per group was sacrificed and the tumors were excised and imaged. Fluorescence imaging settings: Excitation/Emission: 640/730 nm, Excitation Power=10, Binning=2, Exposure time=10 sec, Fstop=2, FOV=25.

Tumor Inoculation and Therapeutic Studies

4T1-Luc tumor cells (1×10⁴) were injected subcutaneously on the right side of the abdomen of BALB/c mice (day 0). Seven days after inoculation, mice were randomized into experimental groups (n=9-10 per group). Mice were intravenously injected via the tail vein with three doses (day 7, 9, 11) or a single dose (only day 7) of CpG (18.2 nmol, 176 ug) or an equal molar amount of 3CM-CpG (18.2 nmol, 250 ug). Mice in the vehicle group were injected IV with sterile PBS (day 7, 9, 11). All treatments were formulated in sterile PBS and filtered (0.2 um sterile filter) prior to injection. Tumor size was monitored with a digital caliper (Mitutoyo) every 2 to 3 days and expressed as volume (length×width×height). Mice were euthanized if tumor size reached 1.5 cm in the largest diameter or if tumors became ulcerated as per guidelines. The recorded survival date indicates when a given mouse reached euthanasia criteria. The Kaplan-Meier method was used for survival analysis. P values were calculated using the log-rank (Mantel-Cox) test.

Synthesis—Functionalized Immunostimulants

Immunostimulants were functionalized with click chemistry handles to enable attachment to targeting agents using strain promoted azide-alkyne cycloaddition (SPAAC). The primary alkyl amine-modified TLR 7/8 agonist (termed T78a) and the 5′ amine-modified Class C CpG-C792 (termed amino-CpG) were purchased from Acme Biosciences and Integrated DNA Technologies, respectively. The immunostimulants were conjugated at sites that were reported to be amendable to modification: N1 linkage for imidazoquinolines and 5′ phosphate for Class C CpG.

The primary alkyl amine of the immunostimulant (amino-CpG or T78a) was functionalized using a N-hydroxysuccinimide (NHS) ester linker to incorporate a bicyclononyne (BCN), dibenzocyclooctyne (DBCO), or azide handle using the respective linkers: BCN-PEG2-NHS ester, DBCO-PEG4-NHS ester, or azide-PEG4-NHS ester (shown in FIG. 2). Amino-CpG (1 eq) was mixed with NHS ester linker (15 eq) in 25% DMSO/75% 100 mM sodium borate buffer (pH 8.5) and was stirred at room temperature overnight. Functionalized CpG was purified from remaining NHS ester linker by size exclusion using Zeba spin desalting columns (7K MWCO) that were buffer exchanged into PBS prior to sample loading. T78a (1.2 eq) was mixed with NHS ester linker (1 eq) in anhydrous DMSO with 4.4 eq triethylamine and was stirred at room temperature for 5-8 hours. Reactions were monitored using analytical HPLC and/or LCMS using an analytical C18 column. HPLC method for monitoring CpG functionalization: Linear gradient from 5% to 65% solvent B over 30 min (solvent A: 100 mM triethylammonium acetate in water pH 7; solvent B: acetonitrile; 35° C.). HPLC method for monitoring T78a functionalization: isocratic hold at 5% solvent B for 2 minutes, followed by a linear gradient from 5% to 75% solvent over 15 min (solvent A: water+0.1% TFA; solvent B: acetonitrile+0.1% TFA; room temperature).

In addition to synthesizing DBCO-CpG, DBCO-CpG was also ordered directly from Integrated DNA Technologies (IDT).

Synthesis—Knottin Peptide Conjugates

Solid phase peptide synthesis (SPPS) was used to synthesize knottin peptides using standard Fmoc conditions. Peptide synthesis, cleavage, folding, and HPLC purification protocols are described in Cox et al. (2016) Angew Chem Int Ed. 55(34): 9894-7. Given the knottin peptides have no lysine residues, 2.5F was site-specifically conjugated at the N-terminal amine using an azide-PEG4-NHS ester linker with 4.4 eq of TEA in DMSO at room temperature stirring overnight to produce N-terminal azide-modified 2.5F, which was purified by RP-HPLC. A modified version of 2.5F, termed 3CM, was synthesized with the unnatural amino acid 5-azido-L-norvaline in position 15 of 2.5F's sequence to provide an alternate conjugation site (shown as X₁ azide in FIG. 3A). In addition, the phenylalanine at position 31 (shown as X₂ in FIG. 3A) was substituted for tyrosine in 3CM to facilitate concentration measurements by UV absorption. However, either amino acid at position 31 can be used without compromising binding affinity.

Azide-bearing knottins (3CM or azido-2.5F) (1.2 eq) were reacted with BCN- or DBCO-modified CpG (1 eq) in PBS at 30° C. overnight to produce knottin-CpG conjugates (3CM-CpG or 2.5F-CpG) as shown in FIG. 4. The knottin-CpG conjugates were purified from unreacted knottin peptide by size exclusion using Zeba spin desalting columns (7K MWCO) that were buffer exchanged into PBS prior to sample loading. To produce 3CM-T78a, BCN-T78a (1.15 eq) was reacted with 3CM (1 eq) in 1:1 DMSO/PBS, stirring at room temperature overnight. The reaction was purified by RP-HPLC on a C18 column using a method starting with an isocratic hold at 5% solvent B for 2 minutes, followed by a linear gradient from 5% to 75% solvent over 30 min (solvent A: water+0.1% TFA; solvent B: acetonitrile+0.1% TFA; room temperature). Product fractions were collected, diluted in water, frozen, and lyophilized. Analytical HPLC and/or LCMS were used to monitor reactions using the same methods listed in the “Functionalized immunostimulants” section for CpG and T78a.

Synthesis—Knottin-Fc Conjugates

The Knottin-Fc (KFc) fusion protein was expressed recombinantly and purified as previously described (B. H. Kwan, et al., J Exp Med. 2017, 214(6): 1679-90). NHS ester labeling was used to functionalize KFc fusions with a clickable handle (BCN group) using the BCN-PEG2-NHS ester linker as shown in FIG. 8. NHS ester reacts with primary amines in the protein (lysine residues and N-terminus) to form stable amide linkages. KFc was mixed with BCN-PEG2-NHS ester linker (6 eq) in 100 mM sodium bicarbonate buffer, pH 8.3 at room temperature for 2 hours. This labeling protocol typically produces 2-3 linkages per KFc. BCN-modified KFc was purified from remaining linker by size exclusion using Zeba spin desalting columns (7K MWCO) that were buffer exchanged into PBS prior to sample loading.

To produce KFc-immunostimulant conjugates, BCN-modified KFc (1 eq) was reacted with azido-T78a (6 eq) in PBS at room temperature stirring overnight. The KFc-T78a conjugate was purified from unreacted azido-T78a by size exclusion using Zeba spin desalting columns (7K MWCO) that were buffer exchanged into PBS prior to sample loading.

For synthesis, the BCN and azide groups can be switched such that KFc is modified with azido-NHS ester linker and conjugated to BCN-T78a. Functionalized CpG can be substituted for functionalized T78a as well. DBCO can also be substituted for BCN at any time.

Immune Cell Infiltration

4T1-luc cells (2×10⁴) were implanted subcutaneously in one side of the abdomen of BALB/c mice and allowed to grow for 9 days. Once tumors had developed (referred to as Day 0 post-treatment), mice were treated IV on Day 0 and Day 2 post-treatment with intravenous (IV) or intratumoral (IT) injections for the following groups (n=3 mice per group): Vehicle IV (PBS), CpG IV (18.2 nmol), 3CM-CpG IV (18.2 nmol), and CpG IT (5.2 nmol). The CpG IT dose (5.2 nmol=50 ug) is the typical IT dose given in mice and is lower than the IV doses. Three days after the first dose, tumors were excised and mechanically dissociated into single-cell suspensions.

Cells were incubated with LIVE/DEAD Fixable Aqua Dead Cell Stain prior to antibody staining. Cells were surface-stained with fluorescently-labeled antibodies in phosphate-buffered saline (PBS), 1% bovine serum albumin, and 0.01% sodium azide, fixed in 2% paraformaldehyde, and analyzed by flow cytometry. Data were stored and analyzed using Cytobank (www.cytobank.org).

For data analysis, cells were gated to include only alive single cells. Myeloid-derived suppressor cells (MDSCs) were characterized as CD11b⁺ GR1⁺. NK cells were characterized as CD3⁻ CD49b⁺. CD8⁺ T cells were characterized as CD3⁺ CD8⁺ CD49b⁻. CD4⁺ T cells were characterized as CD3⁺ CD4⁺ CD49b⁻. CD8⁺ B cells were characterized as CD3⁻ B220⁺ CD49b⁻.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A conjugate, comprising:
a knottin peptide comprising an engineered loop that binds to a cell surface molecule; and
an immunostimulant conjugated to the knottin peptide via a linker.

2. The conjugate of claim 1, wherein the immunostimulant activates a pathogen recognition receptor (PRR).

3. The conjugate of claim 2, wherein the PRR is selected from the group consisting of: a Toll-like receptor (TLR), a RIG-1-like receptor (RLR), a NOD-like receptor (NLR), a C-type lectin receptor (CLR), a cytosolic dsDNA sensor (CDS), a stimulator of interferon genes (STING), and any combination thereof.

4. The conjugate of claim 2, wherein the immunostimulant is a Toll-Like Receptor (TLR) agonist.

5. The conjugate of claim 4, wherein the TLR agonist is a TLR 9 agonist.

6. The conjugate of claim 4 or claim 5, wherein the TLR agonist is an oligonucleotide-based TLR agonist.

7. The conjugate of claim 6, wherein the oligonucleotide-based TLR agonist comprises one or more CpG dinucleotides.

8. The conjugate of claim 7, wherein the oligonucleotide-based TLR agonist is a CpG oligodeoxynucleotide (ODN).

9. The conjugate of claim 8, wherein the CpG ODN is from a class selected from the group consisting of: class A (type D), class B (type K), and class C.

10. The conjugate of claim 4, wherein the TLR agonist is an agonist of TLR 7, TLR8, or both.

11. The conjugate of claim 10, wherein the TLR agonist comprises an imidazoquinoline (IMZQ) compound.

12. The conjugate of claim 11, wherein the TLR agonist is selected from the group consisting of: T78a, Hybrid-2, Para-amine, Meta-amine, XG1-236, DS802, CL075, CL097, and R848.

13. The conjugate of claim 12, wherein the TLR agonist is T78a.

14. The conjugate of any one of claims 1 to 13, wherein the knottin peptide is conjugated to two or more immunostimulants.

15. The conjugate of claim 14, wherein two of the two or more immunostimulants are the same.

16. The conjugate of claim 14 or claim 15, wherein two of the two or more immunostimulants are different.

17. The conjugate of any one of claims 14 to 16, wherein the two or more immunostimulants are independently selected from an immunostimulant as defined in any one of claims 2 to 13.

18. The conjugate of any one of claims 1 to 17, wherein the knottin peptide is further conjugated to a detectable label.

19. The conjugate of claim 18, wherein the detectable label is an in vivo imaging agent.

20. The conjugate of any one of claims 1 to 19, wherein the knottin peptide is fused to one or more heterologous polypeptides.

21. The conjugate of claim 20, wherein the one or more heterologous polypeptides comprises an Fc domain, an albumin, a transferrin, XTEN, a homo-amino acid polymer, a proline-alanine-serine polymer, an elastin-like peptide, or any combination thereof.

22. The conjugate of claim 21, wherein the one or more heterologous polypeptides comprises an Fc domain.

23. The conjugate of claim 22, wherein the Fc domain is a human Fc domain.

24. The conjugate of any one of claims 20 to 23, wherein the one or more heterologous polypeptides comprises a polypeptide detectable in vivo.

25. The conjugate of any one of claims 1 to 24, wherein the knottin peptide is selected from the group consisting of: an EETI-II peptide, an AgRP peptide, a w-conotoxin peptide, a Kalata B1 peptide, an MCoTI-II peptide, an agatoxin peptide, and a chlorotoxin peptide.

26. The conjugate of claim 25, wherein the knottin peptide is an EETI-II peptide.

27. The conjugate of claim 26, wherein the knottin peptide comprises an amino acid sequence having 70% or greater, 80% or greater, 90% or greater, 95% or greater, or 100% identity to the amino acid sequence set forth in SEQ ID NO:8.

28. The conjugate of claim 26, wherein the knottin peptide comprises an amino acid sequence having 70% or greater, 80% or greater, 90% or greater, 95% or greater, or 100% identity to the amino acid sequence set forth in SEQ ID NO:9.

29. The conjugate of claim 26, wherein the knottin peptide comprises an amino acid sequence having 70% or greater, 80% or greater, 90% or greater, 95% or greater, or 100% identity to the amino acid sequence set forth in SEQ ID NO:10.

30. The conjugate of any one of claims 1 to 29, wherein the cell surface molecule is a cancer cell surface molecule.

31. The conjugate of claim 30, wherein the cancer cell surface molecule is present on cancer cells of a solid tumor.

32. The conjugate of claim 30, wherein the cancer cell surface molecule is present on cancer cells of a liquid tumor.

33. The conjugate of any one of claims 1 to 32, wherein the cell surface molecule is a cell surface receptor.

34. The conjugate of claim 33, wherein the cell surface receptor is a cell adhesion receptor.

35. The conjugate of claim 34, wherein the cell adhesion receptor is an integrin.

36. The conjugate of claim 35, wherein the integrin is selected from the group consisting of: αvβ1 integrin, αvβ3 integrin, αvβ5 integrin, αvβ6 integrin, α5β1 integrin, and any combination thereof.

37. The conjugate of any one of claims 34 to 36, wherein the cell adhesion receptor is present on tumor vasculature cells.

38. The conjugate of claim 33, wherein the cell surface receptor is selected from the group consisting of: a growth factor receptor, a chemokine receptor, an immune cell receptor, and combinations thereof.

39. The conjugate of any one of claims 1 to 32, wherein the cell surface molecule is a membrane protease.

40. A composition comprising the conjugate of any one of claims 1 to 39.

41. The composition of claim 40, wherein the composition is a pharmaceutical composition comprising:
the conjugate; and
a pharmaceutically acceptable carrier.

42. The composition of claim 41, wherein the pharmaceutical composition is formulated for parenteral administration.

43. The composition of claim 41, wherein the pharmaceutical composition is formulated for oral administration.

44. A kit comprising:
a therapeutically effective amount of the pharmaceutical composition of any one of claims 41 to 43; and
instructions for administering the pharmaceutical composition to an individual in need thereof.

45. The kit of claim 44, wherein the pharmaceutical composition is present in one or more unit dosages.

46. A method comprising administering a therapeutically effective amount of the pharmaceutical composition of any one of claims 41 to 43 to an individual in need thereof.

47. The method according to claim 46, wherein the administering is by systemic administration.

48. The method according to claim 47, wherein the systemic administration is by parenteral administration.

49. The method according to claim 48, wherein the parenteral administration is by intravenous administration.

50. The method according to claim 47, wherein the systemic administration is by oral administration.

51. The method according to any one of claims 46 to 50, wherein the individual has cancer, the engineered loop binds to a cell surface molecule on cancer cells present in the individual, and the method is a method of treating the cancer of the individual.

52. The method according to claim 51, wherein the cell surface molecule is a cell surface receptor.

53. The method according to claim 52, wherein the cell surface receptor is a cell adhesion receptor.

54. The method according to claim 53, wherein the cell adhesion receptor is an integrin.

55. The method according to claim 54, wherein the integrin is selected from the group consisting of: αvμ1 integrin, αvμ3 integrin, αvμ5 integrin, αvμ6 integrin, α5β1 integrin, and any combination thereof.

56. The method according to any one of claims 51 to 55, wherein the individual has a solid tumor comprising the cancer cells.

57. The method according to claim 56, wherein the administering is by systemic administration, and wherein the immune cell microenvironment of the solid tumor is characterized by one or any combination of increased percentage of CD8+ T cells, increased percentage of CD4+ T cells, increased percentage of B cells, and/or decreased percentage of myeloid-derived suppressor cells (MDSCs), as compared to the immune cell microenvironment of the tumor when the immunostimulant alone is administered systemically to the individual.

58. The method according to claim 56, wherein the administering is by systemic administration, and wherein the immune cell microenvironment of the solid tumor as assessed by one or any combination of the percentage of CD8+ T cells, the percentage of CD4+ T cells, the percentage of B cells, and/or the percentage of myeloid-derived suppressor cells (MDSCs), is not statistically significantly different as compared to the immune cell microenvironment of the tumor when the immunostimulant alone is administered intratumorally to the individual.

59. The method according to any one of claims 51 to 55, wherein the individual has a liquid tumor comprising the cancer cells.

60. A method of making a knottin-immunostimulant conjugate, comprising conjugating an immunostimulant to a knottin peptide via a linker.

61. The method according to claim 60, wherein the conjugating comprises:
functionalizing the immunostimulant; and
conjugating the functionalized immunostimulant to the knottin peptide.

62. The method according to claim 61, wherein the immunostimulant comprises a primary amine, and wherein functionalizing the immunostimulant comprises reacting the primary amine with an amine-reactive linker.

63. The method according to claim 62, wherein the amine-reactive linker is an amine-reactive NHS ester linker.

64. The method according to claim 62 or claim 63, wherein the amine-reactive linker comprises a moiety selected from the group consisting of: bicyclo[6.1.0]nonyne (BCN), dibenzocyclooctyne (DBCO), and an azide moiety.

65. The method according to claim 64, wherein conjugating the functionalized immunostimulant to the knottin peptide comprises reacting the moiety of the amine-reactive linker with a moiety of the knottin peptide.

66. The method according to claim 65, wherein the knottin peptide comprises a non-natural amino acid comprising the moiety of the knottin peptide.

67. The method according to claim 66, wherein the non-natural amino acid is 5-azido-L-norvaline.

68. The method according to claim 65, wherein the moiety of the knottin peptide is an N-terminal amine group.

69. The method according to any one of claims 60 to 68, wherein the immunostimulant is an immunostimulant as defined in any one of claims 2 to 13.

70. The method according to any one of claims 60 to 69, wherein the knottin peptide is a knottin peptide as defined in any one of claims 20 to 39.
